(19) United States
(12) Patent Application Publication
(10) Pub. No.: US 2005/0261331 A1
(43) Pub. Date: Nov. 24, 2005

(54) Substituted pyrrolopyridines

(76) Inventors: Peter Aadal Nielsen, Lund (SE); Thomas Brimert, Lund (SE); Anna Kristofferson, Lund (SE); Tero Linnanen, Lund (SE); Peter Sjo, Lund (SE)

Correspondence Address: FISH & RICHARDSON P.C., P.O BOX 1022, MINNEAPOLIS, MN, 55440-1022, US

(21) Appl. No.: 10/524,626

(22) PCT Filed: Aug. 13, 2003
(86) PCT No.: PCT/SE03/01272

§ 371 Date: Feb. 10, 2005

(30) Foreign Application Priority Data
Aug. 14, 2002 (SE) 0202463-6

(51) Int. Cl. C07D 471/02; A61K 31/4745

(52) U.S. Cl. 514/300; 546/113

(57) ABSTRACT

There are provided novel compounds of formula (I) wherein R¹, R² and R³ are as defined in the specification and pharmaceutically acceptable salts thereof; together with processes for their preparation, compositions containing them and their use in therapy. The compounds are inhibitors of the kinase Itk.

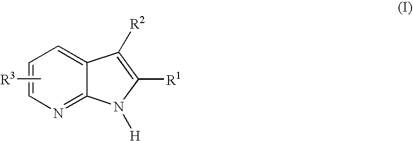

### FIELD OF THE INVENTION

This invention relates to novel 2-heteroaryl- and 2-aryl-7-azaindole[2-(hetero)aryl-1H-pyrrolo[2,3-b]pyridine] derivatives, processes for their preparation, intermediates thereto, pharmaceutical compositions comprising them, and their use in therapy.

### BACKGROUND OF THE INVENTION

Inducible T cell Kinase (Itk) is a member of the Tec-family of cytosolic protein tyrosine kinases. In mammalians, this family also includes Btk, Tec, Bmx, and Txk. These kinases regulate various immune cell functions that integrate signals given by the other cytosolic tyrosine kinases as well as serine/threonine kinases, lipid kinases, and small G proteins. Tec-family kinases have the following general structure: a N-terminal pleckstrin-homology (PH) domain, a Tec-homology domain that includes a Btk motif and one or two proline-rich (PR) motifs, a SH3 domain, a SH2 domain and a c-terminal catalytic (SH1) domain. These kinases are expressed exclusively in hematopoietic tissues, with the exception of Tec and Bmx that have also been detected in endothelial cells. The cellular distribution is different for the Tec-family members. For example, Itk is expressed by T cells, NK cells and mast cells, whereas Btk is expressed by all hematopoietic cells except T cells. Thus, hematopoietic cells may express one or several Tec-family kinases. For example, T cells express Itk, Tec and Txk, and mast cells express Btk, Itk and Tec.

Btk is by far the most extensively studied among the Tec-family kinases, due to its association with X-linked agammaglobulinemia (XLA), and Btk is currently the only Tec-family kinase with a known human phenotype. XLA patients are virtually devoid of mature B cells and their Ig levels are strongly reduced.

Itk^{-/-} mice show defects in T cell activation and differentiation. T helper 2 (Th2) differentiation is disrupted in these mice, whereas Th1 differentiation is apparently intact.

In T and B cells, signalling through T cell receptors and B cell receptors leads to activation of Itk and Btk, respectively. Downstream of Itk and Btk a number of different messengers are engaged; scaffolding proteins (SLP-76, LAT, SLP-65), Src kinases, MAP kinases, and PI3-K. These events are followed by PLC-γ activation that leads to IP3 generation and sustained Ca²⁺ flux, and subsequently activation of transcription factors. PLC-γ1 has been suggested as a direct substrate for Itk.

In T cells, Itk (and Tec) may also mediate signalling through the CD28 co-receptor. Furthermore, Itk has in T cells been implicated in the activation of β-integrin. Signalling from Tec-family kinases can also be regulated by PH domain-mediated plasma membrane localization, and by Src-family-mediated phosphorylation of critical tyrosine residues. Interestingly, Itk, Btk and Txk have recently been shown to translocate to the nucleus after activation.

From studies using Itk−/− mice, it has been proposed that Itk is required for Th2 but not Th1 cell development. This was demonstrated in the N. brasiliensis and L. major infection models where the Itk−/− animals are protected in the Leishmania model indicating an intact Th1 response, whereas they are susceptible to infection with N. Brasiliensis that requires an intact Th2 response for resolution of the infection. This indicates that modulation of Itk activity may prove useful for treatment of Th2-driven disorders and conditions.

We have identified the critical role of Itk in regulating important mast cell and basophil functions and established that the activity of mast cells or basophils may be inhibited through inhibition of Itk. Thus Itk inhibitors may be used as pharmaceutical agents for the treatment of mast cell-driven or basophil-driven conditions or diseases. In particular, we have identified Itk as a target for inhibiting several key events in both acute and late phase allergic reactions common to allergic rhinitis and asthma.

WO 98/22457 discloses aryl and heteroaryl substituted fused pyrrole compounds for the treatment of TNF-α, IL-1β, IL-6 and/or IL-8 mediated diseases.

WO 98/47899 discloses certain 6-substituted 3-(4-pyridyl)-1H-pyrrolo[2,3-b]pyridines and 6-substituted 3-(4-pyrimidyl)-1H-pyrrolo[2,3-b]pyridines as inhibitors of p38 kinase. The compounds are useful in the treatment of diseases associated with the overproduction of inflammatory cytokines. Certain compounds disclosed in this application are disclaimed from the scope of the present invention.

WO 99/20624 discloses certain aza- and diaza- indoles as inhibitors of p38 kinase. However, 7-azaindoles in which N-1 is unsubstituted are not disclosed in this application.

WO 01/47922 discloses substituted aza- and diaza- indoles as kinase inhibitors, in particular, as inhibitors of the protein tryosine kinase Syk.

Henry, J. R. et al., J. Med. Chem. 41 (1998) 4196 describe certain 6-amino-2-(4-fluorophenyl)-3-(4-pyridyl)-1H-pyrrolo[2,3-b]pyridines such as the compound: 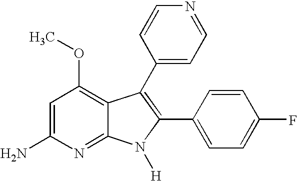
as p38 kinase inhibitors.

The compounds disclosed in J. Med. Chem. 41 (1998) 4196 and in WO 01/47922 are not within the generic scope of the present application.

Henry, J. R. et al., Bioorg. Med. Chem. Letters, 1998, 8, 3335-3340 discloses the compound 2-(4-fluorophenyl)-3-(4-pyridinyl)-1H-pyrrolo[2,3-b]pyridine as a p38 kinase inhibitor.

Patent application JP 11-305996 discloses, inter alia, certain 3-(4-hydroxyphenyl)- and 3-(4-hydroxy-3-pyridyl)-azaindole derivatives. The compounds have activity at the oestrogen receptor and are thereby useful in the treatment of osteoporosis. Certain compounds disclosed in this patent application are disclaimed from the scope of the present invention.

J C S Perkin L 1980, 506-511 discloses the compound 2,3-diphenyl-1H-pyrrolo[2,3-b]pyridine.

J. Chem. Soc. (C) 1969, 1505-1514 discloses the compound 4-methyl-2,3-diphenyl-1H-pyrrolo[2,3-b]pyridine.

None of the above publications are concerned with compounds that have utility as inhibitors of the kinase Itk.

The present invention discloses novel substituted 2-heteroaryl- and 2-aryl-7-azaindoles that have activity as Itk inhibitors and are thereby useful as pharmaceuticals, particularly for the treatment of allergic rhinitis and of asthma.

### DISCLOSURE OF THE INVENTION

The present invention provides a compound of formula (I): 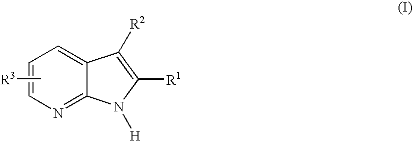
wherein:
R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy, CO₂R⁴ or a group—Q-L-M;
Q represents CO, O; NR¹² or a bond;
L represents C1 to 4 alkyl optionally further substituted by OH or OMe; or L represents a bond;
M represents NR¹³R¹⁴ or OR¹⁵;
R¹³ and R¹⁴ independently represent H, C1 to 4 alkyl or CONH₂; or the group —NR¹³R¹⁴ together represents a saturated 5 to 7 membered azacyclic ring optionally incorporating one further heteroatom selected from O, S and NR¹⁶ and optionally further substituted by OH or 1-piperidinyl;
R¹⁶ represents H, C1 to 4 alkyl, CHO or C2 to 4 alkanoyl;
R² represents phenyl or a five or six membered aromatic heterocyclic ring containing 1 to 3 heteroatoms independently selected from O, S and N; said phenyl or aromatic heterocyclic ring being optionally substituted by one or more substituents selected independently from halogen, C1 to 4 alkyl, C1 to 4 alkoxy, OH, CN, CO₂R⁶ and a group —W—X—Y;
W represents O or a bond;
X represents C1 to 4 alkyl, —CO—, —CH₂CHOHCH₂— or a bond;
Y represents NR⁷R⁸;
or Y represents a saturated or partially unsaturated 4 to 7 membered ring, optionally including 1 or 2 heteroatoms independently selected from O, N and S(O)ₙ and optionally incorporating 1 or 2 carbonyl groups; and optionally substituted by one or more substituents selected independently from OH, C1 to 4 alkyl, C1 to 4 alkoxy, CHO, C2 to 4 alkanoyl, C1 to 4 alkylsulphonyl or CO₂R⁵;
or Y represents C1 to 4 alkoxy optionally further substituted by OH or C1 to 4 alkoxy;
R³ represents H or one or two substituents selected independently from halogen, C1 to 4 alkyl C1 to 4 alkoxy or cyano;
R⁴, R⁵ and R⁶ independently represent H or C1 to 4 alkyl;
R⁷ and R⁸ independently represent H, C1 to 4 alky), —CH₂CHOHCH₂OH, CHO, C2 to 4 alkanoyl or a group -G-J-K wherein G represents —CO— or a bond; J represents C1 to 4 alkyl; and K represents —NR⁹R¹⁰ or —CH(NH₂)CO₂R¹¹;
R⁹ and R¹⁰ independently represent H or C1 to 4 alkyl; or the group —NR⁹R¹⁰ together represents a saturated 5 or 6 membered azacyclic ring;
R¹¹, R¹² and R¹⁵ independently represent H or C1 to 4 alkyl;
n represents an integer 0, 1 or 2;
   and pharmaceutically acceptable salts thereof;
   provided that:
(i) when R³ is at the 6-position and represents C1 to 4 alkoxy and at the same time R¹ represents optionally substituted phenyl, then R² does not represent unsubstituted 4-pyridyl or unsubstituted 4-pyrimidyl; and
(ii) when R² represents 4-hydroxyphenyl or 4-hydroxy-3-pyridyl either optionally further substituted by halogen, C1 to 4 alkyl or C1 to 4 alkoxy, then R³ represents cyano; and
(iii) the following three compounds are disclaimed -2-(4-fluorophenyl)-3-(4-pyridinyl)-1H-pyrrolo[2,3-b]pyridine; 2,3-diphenyl-1H-pyrrolo[2,3-b]pyridine; and 4-methyl-2,3-diphenyl-1H-pyrrolo[2,3-b]pyridine.

The compounds of formula (I) may exist in enantiomeric forms. All enantiomers, diastereoisomers, racemates and mixtures thereof are included within the scope of the invention.

Compounds of formula (I) may also exist in various tautomeric forms. All possible tautomeric forms and mixtures thereof are included within the scope of the invention.

Unless otherwise indicated, the term “C1 to 4 alkyl” referred to herein denotes a straight or branched chain alkyl group having from 1 to 4 carbon atoms. Examples of such groups include methyl, ethyl, n-propyl, i-propyl, n-butyl, i-butyl and t-butyl.

Unless otherwise indicated, the term “C1 to 4 alkoxy” referred to herein denotes an oxygen substituent bonded to a straight or branched chain alkyl group having from 1 to 4 carbon atoms. Examples of such groups include methoxy, ethoxy, n-propoxy, i-propoxy, n-butoxy, i-butoxy and s-butoxy.

Unless otherwise indicated, the term “C2 to 4 alkanoyl” referred to herein denotes a carbonyl group attached to a straight or branched chain alkyl group having from 1 to 3 carbon atoms. Examples of such groups include acetyl and propionyl.

Unless otherwise indicated, the term “C1 to 4 alkylsulphonyl” referred to herein denotes a sulphonyl group, —SO₂—, attached to a straight or branched chain alkyl group having from 1 to 4 carbon atoms. Examples of such groups include methylsulphonyl and ethylsulphonyl.

Unless otherwise indicated, the term “halogen” referred to herein denotes fluorine, chlorine, bromine and iodine.

Examples of a five or six membered aromatic heterocyclic ring containing 1 to 3 heteroatoms independently selected independently from O, S and N include furan, thiophene, pyrrole, pyridine, imidazole, thiazole, oxazole, isoxazole, isothiazole, triazole, oxadiazole; pyrazine and pyrimidine.

Examples of a saturated or partially unsaturated 4 to 7 membered ring, optionally including 1 or 2 heteroatoms independently selected from O, N and S(O)ₙ and optionally incorporating 1 or 2 carbonyl groups include cyclopentane, cyclohexane, cycloheptane, pyrrolidine, piperidine, morpholine, thiomorpholine, piperazine, pyrrolidinone, oxazolidinone, piperidinone, tetrahydrofuran, cyclopentene, dihydroimidazole and dehydropiperidine.

Examples of a saturated 5 to 7 membered azacyclic ring optionally incorporating one further heteroatom selected from O, S and N include pyrrolidine, piperidine, morpholine and piperazine.

In one embodiment, R¹ in formula (I) represents optionally substituted phenyl, furyl, thienyl, thiazolyl, pyrrolyl or oxazolyl. In another embodiment, R¹ represents phenyl, furyl or pyrrolyl, optionally substituted by C1 to 2 alkoxy or halogen.

In one embodiment, R³ in formula (I) represents a single substituent that is located at the 5-position of the azaindole ring system. In another embodiment, R³ in formula (I) represents two independent substituents that are located at the 4- and 5-positions of the azaindole ring system. In one embodiment, R³ represents halogen, methyl, methoxy or cyano. In another embodiment, R³ represents bromo or chloro.

In one embodiment, R² represents phenyl substituted by C1 to 4 alkoxy or by a group —W—X—Y. In another embodiment, R² represents 5-pyrimidinyl.

In another embodiment W in formula (I) represents O.

In one embodiment, the invention provides a compound of formula (Ia) 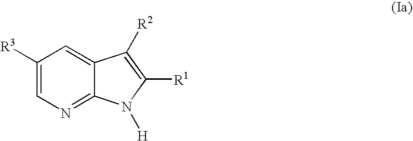
wherein:
R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy or CO₂R⁴;
R² represents phenyl or a five or six membered aromatic heterocyclic ring containing 1 to 3 heteroatoms independently selected from O, S and N; said phenyl or aromatic heterocyclic ring being optionally substituted by one or more substituents selected independently from halogen, C1 to 4 alkyl, C1 to 4 alkoxy, CN, CO₂R⁶ and a group —W—X—Y;
W represents O or a bond;
X represents C1 to 4 alkyl, —CO—, —CH₂CHOHCH₂— or a bond;
Y represents NR⁷R⁸;
or Y represents a saturated or partially unsaturated 4 to 7 membered ring, optionally including 1 or 2 heteroatoms independently selected from O, N and S(O)ₙ and optionally incorporating 1 or 2 carbonyl groups; and optionally substituted by one or more substituents selected independently from OH, C1 to 4 alkyl, C1 to 4 alkoxy, CHO, C2 to 4 alkanoyl, C1 to 4 alkylsulphonyl or CO₂R⁵;
or Y represents C1 to 4 alkoxy optionally further substituted by OH or C1 to 4 alkoxy;
R³ represents one or two substituents independently selected from halogen, C1 to 4 alkyl, C1 to 4 alkoxy or cyano;
R⁴, R⁵ and R⁶ independently represent H or C1 to 4 alkyl;
R⁷ and R⁸ independently represent H, C1 to 4 alkyl, —CH₂CHOHCH₂OH, CHO, C2 to 4 alkanoyl or a group -G-J-K wherein G represents —CO— or a bond; J represents C1 to 4 alkyl; and K represents —NR⁹R¹⁰ or —CH(NH₂)CO₂R¹¹;
R⁹ and R¹⁰ independently represent H or C1 to 4 alkyl; or the group —NR⁹R¹⁰ together represents a saturated 5 or 6 membered azacyclic ring;
R¹¹ represents H or C1 to 4 alkyl;
n represents an integer 0, 1 or 2;
   and pharmaceutically acceptable salts thereof.

Particular compounds according to the present invention include:
5-bromo-3-(4-methoxyphenyl)-2-phenyl-1H-pyrrolo[2,3-b]pyridine;
5-bromo-3-(3-methoxyphenyl)-2-phenyl-1H-pyrrolo[2,3-b]pyridine;
4-(5-bromo-2-phenyl-1H-pyrrolo[2,3-b]pyridin-3-yl)benzonitrile;
5-bromo-2-(2-furyl)-3-phenyl-1H-pyrrolo[2,3-b]pyridine;
3-(4-[5-bromo-2-(2-furyl)-1H-pyrrolo[2,3-b]pyridin-3-yl]phenoxy)-N,N-dimethylpropan-1-amine;
5-bromo-3-(4-morpholin-4-ylphenyl)-2-phenyl-1H-pyrrolo[2,3-b]pyridine;
5-bromo-2,3-diphenyl-1H-pyrrolo[2,3-b]pyridine;
5-bromo-2-(4-bromophenyl)-3-phenyl-1H-pyrrolo[2,3-b]pyridine;
5-bromo-2,3-bis(4-methoxyphenyl)-1H-pyrrolo[2,3-b]pyridine;
N-(3-{4-[5-bromo-2-(2-furyl)-1H-pyrrolo[2,3-b]pyridin-3-yl]phenoxy}propyl)-N,N-dimethylamine;
5-bromo-3-phenyl-2-(1,3-thiazol-2-yl)-1H-pyrrolo[2,3-b]pyridine;
5-bromo-3-furan-2-yl-1-H-pyrrolo[2,3-b]pyridine;
N-[5-(5-bromo-2-phenyl-1-H-pyrrolo[2,3-b]pyridin-3-yl)-fuan-2-ylmethyl]-acetamide;
5-bromo-3-(5-aminomethylfuran-2-yl)-2-phenyl-1H-pyrrolo[2,3-b]pyridine;
5-bromo-2,3-difuran-2-yl-1H-pyrrolo[2,3-b]pyridine;
methyl 5-(5-bromo-3-phenyl-1H-pyrrolo[2,3-b]pyridin-2-yl)-1H-pyrrole-2-carboxylate;
5-bromo-3-phenyl-2-(1H-pyrrol-3-yl)-1H-pyrrolo[2,3-b]pyridine;
5-bromo-3-phenyl-2-(1,3-oxazol-2-yl)-1H-pyrrolo[2,3-b]pyridine;
3-(5-bromo-2-phenyl-1H-pyrrolo[2,3-b]pyridin-3-yl)phenol;
1-[4-(5-bromo-2-phenyl-1H-pyrrolo[2,3-b]pyridin-3-yl)phenoxy]-3-[(2-pyrrolidin-1-ylethyl)amino]propan-2-ol;
1-[4-(5-bromo-2-phenyl-1H-pyrrolo[2,3-b]pyridin-3-yl)phenoxy]-3-pyrrolidin-1-ylpropan-2-ol;
5-bromo-3-{4-[2-(1-methylpyrrolidin-2-yl)ethoxy]phenyl}-2-phenyl-1H-pyrrolo[2,3-b]pyridine;
5-bromo-2-phenyl-3-[4-(2-pyrrolidin-1-ylethoxy)phenyl]-1H-pyrrolo[2,3-b]pyridine;
5-bromo-3-[4-(2-morpholin-4-ylethoxy)phenyl]-2-phenyl-1H-pyrrolo[2,3-b]pyridine;
5-bromo-3-[3-(2-morpholin-4-ylethoxy)phenyl]-2-phenyl-1H-pyrrolo[2,3-b]pyridine;
3-[4-(5-bromo-2-phenyl-1H-pyrrolo[2,3-b]pyridin-3-yl)phenoxy]-N,N-dimethylpropan-1-amine;
5-bromo-3-{4-[2-(2-methoxyethoxy)ethoxy]phenyl}-2-phenyl-1H-pyrrolo[2,3-b]pyridine;
5-bromo-3-{3-[2-(1-methylpyrrolidin-2-yl)ethoxy]phenyl}-2-phenyl-1H-pyrrolo[2,3-:b]pyridine;
3-{4-[3-(dimethylamino)propoxy]phenyl}-2-phenyl-1H-pyrrolo[2,3-b]pyridine-5-carbonitrile;
5-{[4-(5-bromo-2-phenyl-1H-pyrrolo[2,3-b]pyridin-3-yl)phenoxy]methyl}-1,3-oxazolidin-2-one;
3-{4-[3-(dimethylamino)propoxy]phenyl}-2-(4-methoxyphenyl)-1H-pyrrolo[2,3-b]pyridine-5-carbonitrile;
(3-{4-[5-bromo-2-(4-methoxy-phenyl)-1H-pyrrolo[1,3-b]pyridin-3-yl]-phenoxy}-propyl)-dimethylamine;
3-[4-(5-bromo-2-phenyl-1H-pyrrolo[2,3-b]pyridin-3-yl)phenoxy]propan-1-amine;
5-bromo-3-(4-aminomethylphenyl)-2-phenyl-1H-pyrrolo[2,3-b]pyridine;
5-bromo-3-[4-(4,5-dihydro-1H-imidazol-2-yl)phenyl]-2-phenyl-1H-pyrrolo[2,3-b]pyridine;
5-bromo-3-[4-(4,4-dimethyl-4,5-dihydro-1H-imidazol-2-yl)phenyl]-2-phenyl 1H-pyrrolo[2,3-b]pyridine;
N-(2-aminoethyl)-4-(5-bromo-2-phenyl-1H-pyrrolo[2,3-b)pyridin-3-yl)benzamide;
3-[[4-(5-bromo-2-phenyl-1H-pyrrolo[2,3-b]pyridin-3-yl)benzyl](1,2-dihydroxypropyl)amino]propane-1,2-diol;
4-(5-bromo-2-phenyl-1H-pyrrolo[2,3-b]pyridin-3-yl)benzoic acid;
N⁵-[4-(5-bromo-2-phenyl-1H-pyrrolo[2,3-b]pyridin-3-yl)benzyl]glutamine;
3-(4-hydroxyphenyl)-2-phenyl-1H-pyrrolo[2,3-b]pyridine-5-carbonitrile;
3-[4-(aminomethyl)phenyl]-2-phenyl-1H-pyrrolo[2,3-b]pyridine-5-carbonitrile;
3-(4-morpholin-4-ylphenyl)-2-phenyl-1H-pyrrolo[2,3-b]pyridine-5-carbonitrile;
3-(4-hydroxyphenyl)-2-(4-methoxyphenyl)-1H-pyrrolo[2,3-b]pyridine-5-carbonitrile;
5-bromo-2-phenyl-3-pyrrol-1-yl-1H-pyrrolo[2,3-b]pyridine;
5-cyano-2-(4-methoxy-phenyl)-3-pyrrol-1-yl-1H-pyrrolo[2,3-b]pyridine;
5-bromo-3-(2,5-dimethyl-pyrrol-1-yl)-2-phenyl-1H-pyrrolo[2,3-b]pyridine;
3-(4-methoxyphenyl)-2-phenyl-1H-pyrrolo[2,3-b]pyridine-5-carbonitrile;
{3-[4-(5-methyl-3-pyrimidin-5-yl-1H-pyrrolo[2,3-b]pyridin-2-yl)phenoxy]propyl}dimethylamine;
{3-[4-(5-fluoro-3-pyrimidin-5-yl-1H-pyrrolo[2,3-b]pyridin-2-yl)phenoxy]propyl}dimethylamine;
2-{4-[3-(dimethylamino)propoxy]phenyl}-4-methyl-3-pyridin-3-yl-1H-pyrrolo[2,3-b]pyridine-5-carbonitrile;
5-chloro-2-[5-(piperazin-1-ylcarbonyl)-1H-pyrrol-3-yl]-3-pyridin-3-yl-1H-pyrrolo[2,3-b]pyridine;
5-chloro-2-[5-(piperazin-1-ylcarbonyl)-1H-pyrrol-3-yl]-3-pyrimidin-5-yl-1H-pyrrolo[2,3-b]pyridine;
{3-[4-(4,5-dichloro-3-pyrimidin-5-yl-1H-pyrrolo[2,3-b]pyridin-2-yl)phenoxy]propyl}dimethylamine;
{3-[4-(5-bromo-4-methyl-3-pyridin-3-yl-1H-pyrrolo[2,3-b]pyridin-2-yl)phenoxy]propyl}dimethylamine;
5-chloro-3-pyridin-3-yl-2-(1H-pyrrol-2-yl)-1H-pyrrolo[2,3-b]pyridine;
5-chloro-3-pyridin-3-yl-2-(1H-pyrrol-3-yl)-1H-pyrrolo[2,3-b]pyridine;
5-chloro-4-methoxy-3-pyridin-3-yl-2-(1H-pyrrol-3-yl)-1H-pyrrolo[2,3-b]pyridine;
5-chloro-2-(6-chloropyridin-3-yl)-3-pyrimidin-5-yl-1H-pyrrolo[2,3-b]pyridine;
(2-{[5-(5-chloro-3-pyrimidin-5-yl-1H-pyrrolo[2,3-b]pyridin-2-yl)pyridin-2-yl]oxy}ethyl)methylamine;
N-[5-(5-chloro-3-pyrimidin-5-yl-1H-pyrrolo[2,3-b]pyridin-2-yl)pyridin-2-yl]-N,N′,N′-trimethylpropane-1,3-diamine;
N′-[5-(5-chloro-3-pyrimidin-5-yl-1H-pyrrolo[2,3-b]pyridin-2-yl)pyridin-2-yl]-N,N-dimethylpropane-1,3-diamine;
N-{3-[4-(5-chloro-3-pyrimidin-5-yl-1H-pyrrolo[2,3-b]pyridin-2-yl)phenoxy]propyl}-N,N-dimethylamine;
{3-[4-(5-chloro-4-methoxy-3-pyridin-3-yl-1H-pyrrolo[2,3-b]pyridin-2-yl)phenoxy]propyl}dimethylamine;
N-(2-{([5-(5-chloro-3-pyrimidin-5-yl-1H-pyrrolo[2,3-b]pyridin-2-yl)pyridin-2-yl]oxy}ethyl)urea;
2-{[5-(5-chloro-3-pyrimidin-5-yl-1H-pyrrolo[2,3-b]pyridin-2-yl)pyridin-2-yl]oxy}ethanol;
2-[6-(4-acetylpiperazin-1-yl)pyridin-3-yl]-5-chloro-3-pyrimidin-5-yl-1H-pyrrolo[2,3-b]pyridine;
5-chloro-3-(4,5-dihydropyrimidin-5-yl)-2-[6-(4-methylpiperazin-1-yl)pyridin-3-yl]-1H-pyrrolo[2,3-b]pyridine;
5-chloro-3-(4,5-dihydropyrimidin-5-yl)-2-(6-morpholin-4-ylpyridin-3-yl)-1H-pyrrolo[2,3-b]pyridine;
1-[4-(5-chloro-3-pyrimidin-5-yl-1H-pyrrolo[2,3-b]pyridin-2-yl)phenoxy]-3-(4-methylpiperazin-1-yl)propan-2-ol;
1-[4-(5-chloro-1-pyrimidin-5-yl-1H-pyrrolo[2,3-b]pyridin-2-yl)phenoxy]-3-(dimethylamino)propan-2-ol;
1-[4-(5-chloro-3-pyrimidin-5-yl-1H-pyrrolo[2,3-b]pyridin-2-yl)phenoxy]-3-morpholin-4-ylpropan-2-ol;
1-{3-[4-(5-chloro-3-pyrimidin-5-yl-1H-pyrrolo[2,3-b]pyridin-2-yl)phenoxy]-2-hydroxypropyl}pyrrolidin-3-ol;
1-(1,4′-bipiperidin-1′-yl)-3-(4-(5-chloro-3-pyrimidin-5-yl-1H-pyrrolo[2,3-b]pyridin-2-yl)phenoxy]propan-2-ol;
{3-[4-(5-chloro-3-pyrimidin-5-yl-1H-pyrrolo[2,3-b]pyridin-2-yl)phenoxy]-2-methoxypropyl}dimethylamine;
[4-(5-chloro-3-pyrimidin-5-yl-1H-pyrrolo[2,3-b]pyridin-2-yl)phenyl][2-(4-methylpiperazin-1-yl)ethyl]amine;
5-chloro-2-(1H-pyrazol-4-yl)-3-pyridin-3-yl-1H-pyrrolo[2,3-b]pyridine;
5-chloro-2-{4-[3-(dimethylamino)propoxy]phenyl}-N-methyl-3-pyrimidin-5-yl-1H-pyrrolo[2,3-b]pyridin-4-amine;
   and pharmaceutically acceptable salts thereof.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

In a further aspect the invention provides a process for the preparation of a compound of formula (I) which comprises:
a) reaction of a compound of formula (II): 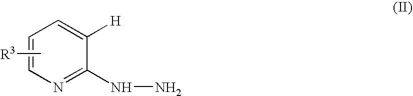
   in which R³ is as defined in formula (I), with a compound of formula (III): 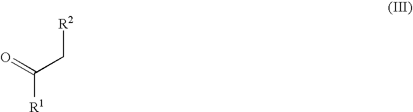
   in which R¹ and R² are as defined in formula (I); or
b) arylation of a compound of formula (IV) 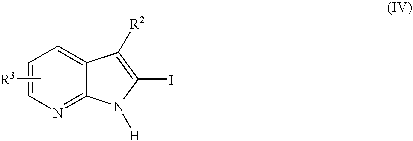
   wherein R² and R³ are as defined above, with a boronic acid of formula R¹—B(OH)₂ wherein R¹ is as defined above;
   and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

Process (a) may be carried out by heating together at a suitable temperature and preferably in an inert atmosphere the compounds of formulae (II) and (III), optionally in the presence of an inert solvent. Preferably the reaction is carried out at a temperature between 100° C. and 250° C., preferably in the absence of a solvent. Suitable reaction times are generally from 5 minutes to 3 hours.

Alternatively process (a) may be carried out in two steps. In the first step, the compounds of formulae (II) and (III) are condensed together to give an intermediate hydrazone of formula (V) 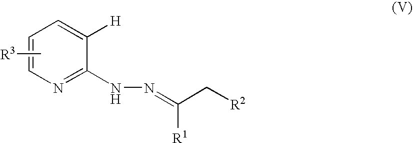
wherein R¹, R² and R³ are as defined in formula (I).

And in a second step the hydrazone (V) is cyclised by heating under similar conditions to those used for the single step process above. The condensation of compounds of formulae (II) and (III) to give the hydrazone (V) is generally carried out in an inert solvent such as benzene or toluene in the presence of an acid catalyst such as acetic acid or p-toluenesulphonic acid with removal of water by azeotropic distillation.

In process (b), the arylation may be performed in the presence of a suitable palladium catalyst using well known cross-coupling conditions such as those described by A. Suzuki, J. Organomet. Chem. 1999, 576, 147-168.

2-Iodo azaindoles of formula (IV) may be prepared, for example, according to the following Scheme:

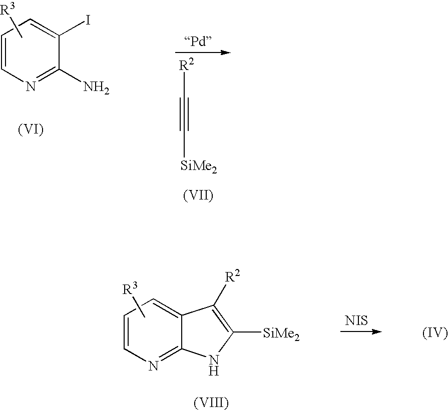

For the cyclization step, conditions as described by F. Ujjainwalla and D. Warner, Tetrahedron Letters, 1998, 39, 5355-5358 may be used. The silyi-iodo-exchange can be performed using N-iodosuccinimide (NIS) according to the protocol described by S. Berteina Raboin et al., Org. Letters, 2002, 4, 2613-2615. Compounds of formula (VI) may, us for example, be obtained by iodination of suitably substituted 2-amino-pyridines using the conditions described by G. A. Olah et al., J. Org. Chem., 1993, 58, 3194-3195.

Aryl boronic acids R¹—B(OH)₂ are either commercially available or may be prepared using well known literature procedures, such as from the corresponding aryl halides.

Compounds of formula (I) in which R¹ represents an aromatic ring substituted by a group—Q-L-M may, when Q represents O, be prepared by alkylation of the corresponding compound wherein the aromatic ring is substituted by OH, using reactions that will be readily apparent to the man skilled in the art. Compounds of formula (I) in which R¹ represents an aromatic ring substituted by a group—Q-L-M may, when Q represents NR¹² be prepared by reductive amination of the corresponding compound wherein the aromatic ring is substituted by NHR¹², using reactions that will be readily apparent to the man skilled in the art. For example,

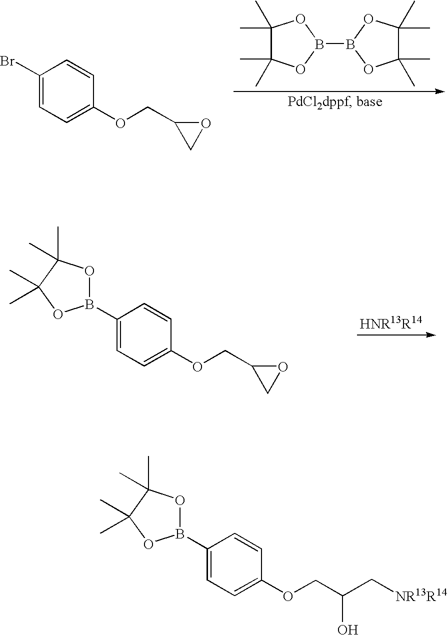

Compounds of formula (I) in which R² represents an aromatic ring substituted by a group —W—X—Y may, when W represents O, be prepared by alkylation of the corresponding compound wherein the aromatic ring is substituted by OH, using reactions that will be readily apparent to the man skilled in the art. Some typical such reactions are illustrated within the Examples disclosed herein.

Alkynes (VII) may be synthezised starting from a suitably protected aldehyde by analogy to the protocol described by K. Miwa, T. Aoyama and T. Shioiri, Synlett., 1994, 107-108.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vacuo or by freeze drying. Suitable solvents include, for example, water, dioxan, ethanol, 2-propanol, tetrahydrofuran or diethyl ether, or mixtures thereof. The reaction may be a metathetical process or it may be carried out on an ion exchange resin.

Compounds of formula (I) and intermediate compounds thereto may be prepared as such or in protected form. The protection and deprotection of functional groups is, for example, described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

The compounds of the invention and intermediates may be isolated from their reaction mixtures, and if necessary further purified, by using standard techniques.

The compounds of formula (I) may exist in enantiomeric or diastereoisomeric forms or mixtures thereof, all of which are included within the scope of the invention. The various optical isomers may be isolated by separation of a racemic mixture of the compounds using conventional techniques, for example, fractional crystallisation or HPLC. Alternatively, the individual enantiomers may be made by reaction of the appropriate optically active starting materials under reaction conditions that will not cause racemisation.

Intermediate compounds may also exist in enantiomeric forms and may be used as purified enantiomers, diastereomers, racemates or mixtures thereof.

According to a further aspect of the invention we provide a compound of formula (I) or a pharmaceutically acceptable-salts thereof, for use as a medicament.

The compounds of formula (I), and their pharmaceutically acceptable salts are useful because they possess pharmacological activity in animals. The compounds of formula (I) have activity as pharmaceuticals, in particular as modulators of kinase activity, especially Itk kinase activity, and as such are predicted to be useful in therapy. They may be used in the treatment or prophylaxis of allergic; autoimmune, inflammatory, proliferative and hyperproliferative diseases and immune-mediated diseases including rejection of transplanted organs or tissues and Acquired Immunodeficiency Syndrome (AIDS).

Thus, another aspect of the invention provides the use of a compound of formula (I) or a pharmaceutically acceptable salt thereof, in the manufacture of a medicament for the treatment or prophylaxis of diseases or conditions in which inhibition of Itk activity is beneficial; and a method of treating, or reducing the risk of, diseases or conditions in which inhibition of Itk activity is beneficial which comprises administering to a person suffering from or at risk of, said disease or condition, a therapeutically effective amount of a compound of formula (I) or a pharmaceutically acceptable salt thereof.

Examples of these conditions are:
(1) (the respiratory tract) airways diseases including chronic obstructive pulmonary disease (COPD) such as irreversible COPD; asthma, such as bronchial, allergic, intrinsic, extrinsic and dust asthma, particularly chronic or inveterate asthma (for example, late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia; sinusitis, chronic rhinosinusitis, nasosinusal polyposis; pulmonary fibrosis;
(2) (bone and joints) rheumatoid arthritis, seronegative spondyloarthropathies (including ankylosing spondylitis, psoriatic arthritis and Reiter's disease), Behcet's disease, Sjogren's syndrome and systemic sclerosis;
(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;
(4) (gastrointestinal tract) Coeliac disease, proctitis, eosinopilic gastro-enteritis, mastocytosis, Crohn's disease, ulcerative colitis, food-related allergies which have effects remote from the gut, for example, migraine, rhinitis and eczema;
(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary to syndrome and idiopathic thrombocytopenia pupura; tuberculosis;
(6) (allograft rejection) acute and chronic following, for example, transplantation of kidney, heart, liver, lung, bone marrow, skin and cornea; and chronic graft versus host disease.

We are particularly interested in Th2-driven and/or mast cell-driven and/or basophil-driven conditions or diseases.

Thus, a more particular aspect of the invention provides the use of a compound of formula (I) or a pharmaceutically acceptable salt thereof, in the manufacture of a medicament for the treatment or prophylaxis of Th2-driven and/or mast cell-driven and/or basophil driven diseases or conditions; and a method of treating, or reducing the risk of, Th2-driven and/or mast cell-driven and/or basophil driven diseases or conditions which comprises administering to a person suffering from or at risk of, said disease or condition, a therapeutically effective amount of a compound of formula (I) or a pharmaceutically acceptable salt thereof.

In a preferred aspect of the invention, we provide a method for the treatment or prevention of a reversible obstructive airway disease, especially asthma, which comprises administering a therapeutically effective amount of a compound of formula (I) or a pharmaceutically acceptable salt thereof to a human that is suffering from or susceptible to the disease. We also provide the use of a compound of formula (I) or a pharmaceutically acceptable salt thereof in the manufacture of a medicament for the treatment or prevention of a reversible obstructive airway disease, especially asthma.

In another preferred aspect of the invention, we provide a method for the treatment or prevention of rhinitis which comprises administering a therapeutically effective amount of a compound of formula (I) or a pharmaceutically acceptable salt thereof to a human that is suffering from or susceptible to rhinitis, especially allergic rhinitis. We also provide the use of a compound of formula (I) or a pharmaceutically acceptable salt thereof in the manufacture of a medicament for the treatment or prevention of rhinitis, especially allergic rhinitis.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dose of the compound to be administered will depend on the compound employed, the disease being treated, the mode of administration, the age, weight and sex of the patient. Such factors may be determined by the attending physician. However, in general, satisfactory results are obtained when the compounds are administered to a human at a daily dosage of between 0.1 mg/kg to 100 mg/kg (measured as the active ingredient).

The compounds of formula (I) may be used on their own, or in the form of appropriate pharmaceutical formulations comprising the compound of the invention in combination with a pharmaceutically acceptable diluent, adjuvant or carrier. Particularly preferred are compositions not containing material capable of causing an adverse reaction, for example, an allergic reaction. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I) in admixture with a pharmaceutically acceptable diluent or carrier.

We also provide a method of preparation of such pharmaceutical formulations that comprises mixing the ingredients.

The compounds may be administered topically, for example, to the lungs and/or the airways, in the form of solutions, suspensions, HFA aerosols or dry powder formulations, for example, formulations in the inhaler device known as the Turbuhaler®; or systemically, for example, by oral administration in the form of tablets, pills, capsules, syrups, powders or granules; or by parenteral administration, for example, in the form of sterile parenteral solutions or suspensions; or by rectal administration, for example, in the form of suppositories.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound with a carrier substance, for example, a mono-, di- or polysaccharide, a sugar alcohol, or another polyol. Suitable carriers are sugars, for example, lactose, glucose, raffinose, melezitose, lactitol, maltitol; trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient, With this system the active compound, with or without a carrier substance, is delivered to the patient.

For oral administration the active compound may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum, titanium dioxide, and the like. Alternatively, the tablet may be coated with a suitable polymer dissolved in a readily volatile organic solvent.

For the preparation of soft gelatine capsules, the compound may be admixed with, for example, a vegetable oil or polyethylene glycol. Hard gelatine capsules may contain granules of the compound using either the above mentioned excipients for tablets. Also liquid or semisolid formulations of the drug may be filled into hard gelatine capsules.

Liquid preparations for oral application may be in the form of syrups or suspensions, for example, solutions containing the compound, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The following Examples are intended to illustrate, but in no way limit the scope of the invention.

General methods All reactions were performed in dried glassware in an argon atmosphere at room temperature, unless otherwise noted. All reagents and solvents were used as is received. Merck Silica gel 60 (0.040-0.063 mm) was used for preparative silica gel chromatography. A Kromasil KR-100-5-C18 column (250×20 mm, Akzo Nobel) and mixtures of acetonitrile/water at a flow rate of 10 ml/min was used for preparative HPLC. Reactions were monitored at 254 nm by analytical HPLC, using a Kromasil C-18 column (150×4.6 mm) and a gradient (containing 0.1% trifluoroacetic acid) of 5 to 100% of acetonitrile in water at a flow rate of 1 ml/min. Evaporations of solvents were performed under reduced pressure using a rotary evaporator at a maximum temperature of 60° C. Products were dried under reduced pressure at about 40° C.

¹H-NMR spectra were recorded on a Varian Inova 400 MHz or Varian Mercury 300 MHz instrument. The central solvent peak of chloroform-d (δ_{H} 7.27 ppm), dimethylsulfoxide-d₆ (δ_{H}2.50 ppm) or methanol-d₄ (δ_{H} 3.35 ppm) were used as internal references. Low resolution mass spectra were obtained on a Hewlett Packard 1100 LC-MS system equipped with a APCI ionisation chamber.

### Preparation 1 N-(5-Bromo-2-phenyl-1H-pyrrolo[2,3-b]pyridin-3-yl)-acetamide

The title compound (430 mg, 26%) was synthesized from N-(2-oxo-2-phenyl-ethyl)-acetamide (900 mg, 5 mmol) and (5-bromo-pyridin-2-yl)-hydrazine (900 mg, 5 mmol) essentially as described in Example 1.

¹H-NMR (DMSO-d₆): δ 12.20 (1H, s); 9.56 (1H, s), 8.29 (1H, s); 7.93 (1H, s); 7.82 (2H, d); 7.50 (2H, t); 7.39 (1H, t); 2.09 (3H, s).

APCI-MS m/z: 330 [MH⁺].

### Preparation 2 N-(5-Cyano-2-phenyl-1H-pyrrolo[2,3-b]pyridin-3-yl)-acetamide

6-Chloro-nicotinonitrile (1.38 g, 10 mmol) was dissolved in 1,4-dioxane (50 ml). Hydrazine hydrate (0.525 ml, 10.4 mmol) was added and the resulting solution stirred for 1.5 h, whereupon it was concentrated in vacuo. The residue was chromatographed (silica gel, gradient ethyl acetate/heptane from 1:1 to 1:0). The slower running component was concentrated in vacuo to afford the 6-hydrazino-nicotinonitrile monohydrate [0.80 g, 53%, is APCI-MS m/z: 135.2 [MH+]]. Part of this hydrazine (67 mg, 0.5 mmol) and N-(2-oxo-2-phenyl-ethyl)-acetamide (85 mg, 0.5 mmol) were fused together for 1 h at 230° C. The reaction mixture was allowed to cool and the glassy solid suspended in warm dichloromethane/methanol (7:3 mixture) and then filtered. The solid was further washed with hot acetonitrile/N,N-dimethylformamide (9:1 mixture) and finally acetonitrile. This afforded the title compound as a grey powder (25 mg, 18%).

¹H-NMR (DMSO-d₆): δ 12.64 (1H, s); 9.66 (1H, s); 8.62 (1H, s); 8.27 (1H, s); 7.84 (2H, d); 7.52 (2H, t); 7.42 (1H, t); 2.10 (3H, s).

¹³C-NMR (DMSO-d₆): δ 147.3; 145.8; 134.0; 131.5; 129.9; 128.8; 128.7; 127.5; 118.8; 117.6; 110.1; 99.9; 22.7.

APCI-MS m/z: 277.1 [MH⁺].

### Preparation 3 5-Bromo-2-phenyl-1H-pyrrolo[2,3-b]pyridin-3-ylamine

N-(5-Bromo-2-phenyl-1H-pyrrolo[2,3-b]pyridin-3-yl)-acetamide (200 mg, 0.6 mmol) was suspended in concentrated hydrochloric acid (20 ml) and heated to reflux overnight. The reaction mixture was allowed to cool to ambient temperature and the precipitate was collected by filtration. This solid was again suspended in water (20 ml) and treated with saturated aqueous sodium hydrogen carbonate until the suspension was neutral. The precipitate was isolated by filtration and thoroughly washed with water to yield the title compound as a yellow powder (170 mg, 97%).

¹H-NMR (DMSO-d₆): δ 12.36 (1H, s); 9.60 (2H, bs); 8.59 (1H, s); 8.34 (1H, s); 7.85 (2H, d); 7.54 (2H, t); 7.45 (1H, t).

APCI-MS m/z: 288.2/292.2 [M⁺].

### EXAMPLE 1

### 5-Bromo-3-(4-methoxyphenyl)-2-phenyl-1H-pyrrolo[2,3-b]pyridine

Under an inert atmosphere 2-(4-methoxyphenyl)-1-phenylethanone (3.16 g, 13.9 mmol) and 5-bromo-2-hydrazinopyridine (2.62 g, 13.9 mmol) were fused together at 230° C. for 70 minutes. After cooling the crude product was crystallized from acetonitrile to give the title compound (3.05 g, 58%).

¹H-NMR (DMSO-d₆): δ 11.60-12.80 (1H, bs); 8.29 (1H, d); 7.89 (1H, d); 7.44-7.48 (2H, m); 7.28-7.37 (3H, m); 7.21 (2H, d); 6.94 (2H, d); 3.87 (3H, s).

APCI-MS m/z: [MH⁺).

Following the general method of Example 1, the compounds of Examples 2 to 11 were prepared:

### EXAMPLE 2

### 5-Bromo-3-(3-methoxyphenyl)-2-phenyl-1H-pyrrolo[2,3-b]pyridine

The title compound (1.62 g, 36%) was synthesized from 2-(3-methoxyphenyl)-1-phenylethanone (2.72 g, 12.0 mmol) and 5-bromo-2-hydrazinopyridine (2.26 g, 12.0 mmol).

¹H-NMR (DMSO-d₆): δ 11.00-13.00 (2H, bs); 8.32 (1H, d); 7.98 (1H, d); 7.42-7.51 (2H, m); 7.23-7.41 (5H, m); 6.81-6.92 (3H, m); 3.65 (3H, s).

### EXAMPLE 3

### 4-(5-Bromo-2-phenyl-1H-pyrrolo[2,3-b]pyridin-3-yl)benzonitrile

The title compound (1.98 g, 35%) was synthesized from 4-(2-oxo-2-phenylethyl)benzonitrile (3.46 g, 15.6 mmol) and 5-bromo-2-hydrazinopyridine (2.86 g, 15.2 mmol).

¹H-NMR (DMSO-d₆): δ 12.60 (1H, s); 8.36 (1H, d); 8.11 (1H, d); 7.82 (2H, d); 7.51 (2H, d); 7.38-7.48 (5H, m).

APCI-MS m/z: 374.1/376.0 [MH⁺].

### EXAMPLE 4

### 5-Bromo-2-(2-furyl)-3-phenyl-1H-pyrrolo[2,3-b]pyridine

The title compound (416 mg, 25%) was synthesized from 112-furyl)-2-phenylethanone (884 mg, 5.0 mmol) and 5-bromo-2-hydrazinopyridine (942 mg, 5.0 mmol).

¹H-NMR (DMSO-d₆): δ 12.50 (1H, d); 8.32 (1H, d); 7.89 (1H, d); 7.63 (1H, d); 7.44-7.48 (5H, m); 6.78 (1H, d); 6.59 (1H, dd).

APCI-MS m/z: 339.0/341.0 [MH⁺].

### EXAMPLE 5

### 3-{4-[S-Bromo-2-(2-furyl)-1H-pyrrolo[2,3-b]pyridin-3-yl]phenoxy}-N,N-dimethylpropan-1-amine trifluoroacetate

The title compound (2.9 mg, 0.6%) was synthesized from N-(2-oxo-2-phenyl-ethyl)-acetamide (253 mg, 0.89 mmol) and (5-bromo-pyridin-2-yl)-hydrazine (165 mg, 0.88 mmol) and purified by preparative HPLC (RP-18, acetonitrile/water/trifluoroacetic acid gradient from 10:90:0.1 to 95:5:0.1).

APCI-MS m/z: 440.1/442.1 [MH⁺].

### EXAMPLE 6

### 5-Bromo-3-(4-morpholin-4-ylphenyl)-2-phenyl-1H-pyrrolo[2,3-b]pyridine

The title compound (67 mg, 29%) was synthesized from 2-(4-morpholin-4-ylphenyl)-1-phenylethanone (150 mg, 0.53 mmol) and (5-bromo-pyridin-2-yl)-hydrazine (100 mg, 0.53 mmol).

¹H-NMR (DMSO-d₆): δ 12.21 (1H, s); 8.24 (1H, d); 7.88 (1H, d); 7.27-7.44 (7H, m); 6.90 (2H, d); 3.70 (4H, dd); 3.14 (4H, dd).

APCI-MS m/z: 434.1/436.1 [MH⁺].

### EXAMPLE 7

### 5-Bromo-2,3-diphenyl-1H-pyrrolo[2,3-b]pyridine

The title compound (68 mg, 37%) was synthesized from 1,2-diphenylethanone (104 mg, 0.53 mmol) and (5-bromo-pyridin-2-yl)-hydrazine (100 mg, 0.53 mmol).

¹H-NMR (DMSO-d₆): δ 12.38 (1H, bs); 8.34 (1H, d); 7.98 (1H, d); 7.48 (2H, dd); 7.29-7.43 (8H, m).

APCI-MS m/z: 349.0/351.0 [MH⁺].

### EXAMPLE 8

### 5-Bromo-2-(4-bromophenyl)-3-phenyl]-1H-pyrrolo[2,3-b]pyridine

The title compound (89 mg, 39%) was synthesized from 1-(4-bromophenyl)-2-phenylethanone (146 mg, 0.53 mmol) and (5-bromo-pyridin-2-yl)-hydrazine (100 mg, 0.53 mmol).

¹H-NMR (DMSO-d₆): δ 12.36 (1H, bs); 8.36 (1H, d); 7.99 (1H, d); 7.60 (2H, d); 7.31-7.45 (7H, m).

APCI-MS m/z: 426.1/427.1/428.1/429.1 [MH⁺].

### EXAMPLE 9

### 5-Bromo-2,3-bis(4-methoxyphenyl)-1H-pyrrolo[2,3-b]pyridine

The title compound (66 mg, 30%) was synthesized from 1,2-bis(4-methoxyphenyl)ethanone (136 mg, 0.53 mmol) and (5-bromo-pyridin-2-yl)-hydrazine (100 mg, 0.53 mmol).

¹H-NMR (DMSO-d₆): δ 12.22 (1H, bs); 8.28 (1H, d); 7.88 (1H, d); 7.42 (2H, d); 7.25 (2H, d); 6.92-7.01 (4H, m); 3.28-3.36 (6H, m).

APCI-MS m/z: 349.0/351.0 [MH+].

### EXAMPLE 10

### N-(3-{4-[5-Bromo-2-(2-furyl)-1H-pyrrolo[2,3-b]pyridin-3-yl]phenoxy}propyl)-N,N-dimethylamine trifluoroacetate

The title compound (4.0 mg, 1.0%) was synthesized from 2-{4-[3-(dimethylamino)propoxy]phenyl}-1-(2-furyl)ethanone (239 mg, 0.83 mmol) and 6-hydrazinonicotinonitrile (111 mg, 0.83 mmol) and purified by preparative HPLC (RP-18, acetonitrile/water/trifluoroacetic acid gradient from 10:90:0.1 to 95:5:0.1).

APCI-MS m/z: 387.2 [MH⁺].

### EXAMPLE 11

### 5-Bromo-3-phenyl-2-(1,3-thiazol-2-yl)-1H-pyrrolo[2,3-b]pyridine

The title compound (51 mg, 6%) was synthesized from 1-(1,3-thiazol-2-yl)-2-phenylethanone (470 mg, 2.3 mmol) and 5-bromo-2-hydrazinopyridine (439 mg, 2.3 mmol).

¹H-NMR (DMSO-d₆): δ 8.41 (1H, d); 7.96 (1H, d); 7.88 (1H, d); 7.73 (1H, d); 7.56-7.46 (5H, m); 7.1 (1H, br s).

APCI-MS m/z: 355.9/357.9 [MH⁺].

### EXAMPLE 12

### 5-Bromo-3-furan-2-yl-2-phenyl-1-H-pyrrolo[2,3-b]pyridine

(5-Bromo-pyridin-2-yl)-hydrazine (1.96 g, 10 mmol) and 2-furan-2-yl-1-phenylethanone (2.05 g, purity 86%, 9.5 mmol) in benzene (40 mL) containing p-toluenesulfonic acid (50 mg) was heated at reflux temperature. Water was continuously distilled off using a Dean-Stark trap. After 16 h, the reaction mixture was cooled, dichloromethane was added and the mixture was washed with saturated aqueous sodium hydrogen carbonate, brine and evaporated. Crystallization from diethyl ether-heptane gave N-(5-bromopyridin-2-yl)-N′-(2-furan-2-yl-1-phenylethylidene)-hydrazine (1.86 g, 55%). M.p. 105-107° C.

¹H-NMR (DMSO-d₆): δ 10.32 (1H, s); 8.24 (1H, dd); 7.87-7-82 (3H, m); 7.51 (1H, dd); 7.41-7.36 (2H, m); 7.35-7.29 (2H, m); 6.33 (1H, dd); 6.18 (1H, dd); and 4.35 (2H, s) ppm. APCI-MS m/z: 356.1/358.1 [MH⁺].

N-(5-Bromopyridin-2-yl)-N′-(2-furan-2-yl-1-phenylethylidene)-hydrazine (440 mg, 1.14 mmol) was stirred in an inert atmosphere at 225° C. for 10 minutes. The crude product was purified with column chromatography (silica gel, ethyl acetate/heptane 1:3) to give the title compound (27 mg, 6.4%) and a second fraction containing additional, slightly impure material (42 mg).

¹H-NMR (DMSO-d₆): δ 12.52 (1H, s); 8.37 (1H, d), 8.28 (1H, d); 7.68 (1H, dd); 7.63-7.58 (2H, m); 7.53-7.44 (3H, m); 6.55 (1H, dd); 6.45 (1H, dd). APCI-MS m/z: 339.1/341.1 [MH⁺].

### EXAMPLE 13

### N-[5-(5-Bromo-2-phenyl-1-H-pyrrolo[2,3-b]pyridin-3-yl)-furan-2-ylmethyl]-acetamide

C-(2,5-Dimethoxy-2,5-dihydrofuran-2-yl)-methylamine (5 g, 31.6 mmol) was acetylated by stirring in a mixture of acetic anhydride (15 mL) and pyridine (25 mL) at ambient temperature for 18-h. Repeated co-evaporation with toluene and triturating the residue with diethyl ether gave N-(2,5-dimethoxy-2,3-dihydrofuran-2-ylmethyl)-acetamide (2.0 g, 31%).

¹H-NMR (CDCl₃): δ 7.28 (1H, s); 6.09 (1H, dd); 5.92 (1H, dd); 5.80-5.70 (1H, b); 5.50 (1H, t); 3.64 (1H, dd); 3.53 (3H, s); 3.44 (1H, dd); 3.23 (3H, s); 1.99 (3H, s).

A mixture of ethyl 3-oxo-3-phenylpropionate (1.88 g, 9.8 mmol) and zinc chloride (0.94 g, 6.9 mmol) in acetic acid (0.41 mL) and water (1.88 mL) was heated at 110° C. Then N-(2,5-dimethoxy-2,5-dihydrofuran-2-ylmethyl)-acetamide (1.91 g, 9.5 mmol) was added in portions during 5 minutes. The reaction mixture was stirred for an additional 5 minutes at 110° C. and was then cooled and partitioned between toluene (20 mL) and water (20 mL). The organic phase was washed with water and brine and then evaporated. Chromatography (silica gel, ethyl acetate-heptane 3:1) gave a mixture (2.42 g) of 2-[5-(acetylaminomethyl)-furan-2-yl]-3-oxo-3-phenylpropionic acid ethyl ester and the corresponding carboxylic acid as an oil. A mixture of this material (2.3 g), lithium chloride (8.85 g), acetic acid (0.7 mL) in N-methylpyrrolidinone (2.1 mL) was stirred at reflux temperature for 22 h. The reaction mixture was then diluted with ethyl acetate, washed twice with water and evaporated. Triturating the residue with diethyl ether gave N-[5-(2-oxo-2-phenylethyl)-furan-2-ylmethyl]-acetamide (0.98 g, 42%).

¹H-NMR (DMSO-d₆): δ 8.26 (1H, bt); 8.02 (2H, d); 7.66 (1H, tt); 7.54 (2H, t); 6.21 (1H, d); 6.16 (1H, d); 4.43 (2H, s); 4.18 (2H, d); 1.82 (3H, s).

APCI-MS m/z: 258.2 [MH⁺].

(5-Bromo-pyridin-2-yl)-hydrazine (0.75 g, 4 mmol) and N-[5-(2-oxo-2-phenylethyl)-furan-2-ylmethyl]-acetamide (0.98 g, 3.8 mmol) in benzene (40 mL) containing p-toluenesulfonic acid (50 mg) was heated at reflux temperature. Water was continuously distilled off using a Dean-Stark trap. After 2 h, the reaction mixture was cooled, toluene was added and the mixture was washed with saturated aqueous sodium hydrogen carbonate solution, water and brine. Evaporation and crystallisation of the residue from ethyl acetate gave the N-(5-{2-[(5-bromopyridin-2-yl)-hydrazono]-2-phenylethyl}-furan-2-ylmethyl)-acetamide (0.77 g, 47%). M.p. 176-176.5° C.

¹H-NMR (DMSO-d₆+D₂O): δ 8.28 (1H, bt); 8.20 (1H, d); 7.85-7.78 (3H, m); 7.41-7.27 (4H, m); 6.08 (1H, d); 5.99 (1H, d); 4.25 (2H, s); 4.12 (2H, s) and 1.78 (3H, s).

APCI-MS m/z: 427.2/429.2 [MH⁺].

N-(5-{2-[(5-Bromopyridin-2-yl)-hydrazono]-2-phenylethyl}-furan-2-ylmethyl)-acetamide (522 mg, 1.22 mmol) was stirred in an inert atmosphere at 225° C. for 16 minutes, cooled and then triturated with ethyl acetate to give the title compound (151 mg, 30%).

¹H-NMR (DMSO-d₆): δ 12.49 (1H, s); 8.37-8.29 (2H, m); 7.70-7.00 (1H, b); 7.68-7.62 (2H, m); 7.53-7.43 (3-H, m); 6.29 (1H, d); 6.26 (1H, d); 4.27 (2H, d); 1.86 (3H, s).

APCI-MS m/z: 410.1/412.1 [MH⁺].

### EXAMPLE 14

### 5-Bromo-3-(5-aminomethylfuran-2-yl)-2-phenyl-1H-pyrrolo[2,3-b]pyridine

A mixture of N-[5-(5-bromo-2-phenyl-1-H-pyrrolo[2,3-b]pyridin-3-yl)-furan-2-ylmethyl]-acetamide (75 mg, 0.18 mmol), methanol (10 mL) and aqueous potassium hydroxide (10 mL, 3M) was refluxed overnight. The methanol was evaporated off and the precipitate was washed with water by repeated centrifugations and dried to give crude title compound (purity 91%) which was further purified by preparative HPLC (RP-18, acetonitrile/water/trifluoroacetic acid gradient from 10:90:0.1 to 95:5:0.1). Acetonitrile was evaporated and the resulting aqueous emulsion was made alkaline with saturated aqueous sodium hydrogen carbonate and extracted three times with dichloromethane. Evaporation of the dichloromethane at reduced pressure gave the title compound as a yellow solid (16 mg, 23%).

¹H-NMR (DMSO-d₆): δ 8.35 (1H, d); 8.30 (1H, d); 7.66-7.62 (2H, m); 7.51-7.42 (3H, m); 6.31 (1H, d); 6.25 (1H, bd); 3.67 (2H, s).

¹³C-NMR (DMSO-d₆): δ 156.2; 146.5; 146.2; 143.4; 136.7; 131.1; 129.5; 128.70; 128.65 (2C); 128.3 (2C); 120.4; 111.5; 106.9; 106.3; 102.1; 38.8.

APCI-MS m/z: 369.1/371.1 [MH⁺]; 351.1/353.1 [MH⁺ —NH₂].

### EXAMPLE 15

### 5-Bromo-2,3-difuran-2-yl-1H-pyrrolo[2,3-b]pyridine

1,2-Di-furan-2-yl-ethanone (1.02 g, 5.8 mmol) and (5-bromo-pyridin-2-yl)-hydrazine (1.09 g, 5.8 mmol) in benzene (40 mL) containing acetic acid (0.4 mL) was heated at reflux temperature for 20 h. Water was continuously distilled off using a Dean-Stark trap. Crude, impure title compound was crystallized from the reaction mixture at 8° C. This material (505 mg) was heated in an inert atmosphere at 230° C. for 7 minutes and then partitioned between toluene and water. The toluene phase was washed with water and brine and then evaporated. The residue was chromatographed (silica gel; ethyl acetate-heptane 1:3) to give the title compound (47 mg, 2.5%).

¹H-NMR (DMSO-d₆): δ 12.59 (1H, s); 8.36 (1H, d); 8.30 (1H, d); 7.91 (1H, dd); 7.80 (1H, dd); 7.10 (1H, dd); 6.84 (1H, dd); 6.71 (1H, dd); 6.64 (1H, dd).

APCI-MS m/z: 321.1/331.1 [MH⁺].

### EXAMPLE 16

### Methyl 5-(5-bromo-3-phenyl-1H-pyrrolo[2,3-b]pyridin-2-yl)-1H-pyrrole-2-carboxylate

(5-Bromo-pyridin-2-yl)-hydrazine (378 mg, 2 mmol) and 4-phenylacetyl-1H-pyrrole-2-carboxylic acid methyl ester (488 mg, 2 mmol) were fused together at 220° C. for 1 h. After cooling the crude product was crystallized from acetonitrile and further purified by preparative HPLC (RP-18, acetonitrile/water/trifluoroacetic acid gradient from 10:90:0.1 to 95:5:0.1) to give the title compound (5 mg, 0.6%).

¹H-NMR (DMSO-d₆): δ 12.17 (1H, bs); 12.13 (1H, bs); 8.22 (1H, s); 7.74 (1H, s); 7.50-7.36 (5H, m); 7.22 (1H, s); 6.94 (1H, s); 3.73 (3H, s).

APCI-MS m/z: 396.3 [MH⁺].

### EXAMPLE 17

### 5-Bromo-3-phenyl-2-(1H-pyrrol-3-yl)-1H-pyrrolo[2,3-b]pyridine

(5-Bromo-pyridin-2-yl)-hydrazine (378 mg, 2 mmol) and 4-phenylacetyl-1H-pyrrole-2-carboxylic acid methyl ester (488 mg, 2 mmol) were fused together at 220° C. for 1 h. After cooling the crude product was crystallized from acetonitrile, purified by preparative HPLC (RP-18, acetonitrile/water/trifluoroacetic acid gradient from 10:90:0.1 to 95:5:0.1) and further chromatographed (silica gel, ethyl acetate/heptane 1:1) to give the title compound (3 mg, 0.4%).

¹H-NMR (DMSO-d₆): δ 11.95 (1H, s); 11.02 (1H, bs); 8.16 (1H, s); 7.69 (1H, s); 7.50-7.40 (4H, m); 7.35 (1H, m); 7.08 (1H, d); 6.73 (1H, m); 6.19 (1H, t).

APCI-MS m/z: 338.1 [MH⁺].

### EXAMPLE 18

### 5-Bromo-3-phenyl-2-(1,3-oxazol-2-yl)-1H-pyrrolo[2,3-b]pyridine

Oxazole (1.6 ml, 24.3 mmol) was dissolved in dry tetrahydrofuran (60 ml). Butyl lithium (1.6M in hexane, 14.5 ml) was slowly added at −25° C., after which the temperature was allowed to rise to 0° C. TMSOTf (4.19 ml, 23.2 mmol) was slowly added and the mixture stirred at room temperature for 20 minutes. Phenylacetylchloride (3.06 ml, 23.1 mmol) was slowly added and the mixture stirred for 3.5 h. Water (20 ml) was added, and the mixture was extracted with dichloromethane. Drying (Na₂SO₄) and evaporation delivered crude material which was purified by column chromatography (silica gel, dichloromethane), affording the 1-(1,3-oxazol-2-yl)-2-phenylethanone as a yellow oil (0.631 g, 14%).

1H-NMR (CDCl₃): δ 7.83 (1H, d); 7.40-7.25 (6H, m); 4.38 (2H, s).

APCI-MS m/z: 188 [MH⁺].

1-(1,3-Oxazol-2-yl)-2-phenylethanone (631 mg, 3.4 mmol) and 5-bromo-2-hydrazinopyridine (634 mg, 3.4 mmol) were fused together at 220° C. for 1 h to give the title compound (332 mg, 29%).

¹H-NMR (DMSO-d₆): δ 12.93 (1H, s); 8.46 (1H, d); 8.21 (1H, s); 8.04 (1H, d); 7.57-7.37 (6H, m).

APCI-MS m/z: 339.9/341.9 [MH⁺].

### EXAMPLE 19

### 3-(5-Bromo-2-phenyl-1H-pyrrolo[2,3-b]pyridin-3-yl)phenol

5-Bromo-3-(3-methoxyphenyl)-2-phenyl-1H-pyrrolo[2,3-b]pyridine (1.41 g, 3.72 mmol) and concentrated aqueous HBr (30 ml) were heated at 120° C. for 64 h under an inert atmosphere. After cooling and basification with aqueous ammonia the product was filtered off, washed with water and dried in vacuo to give the title compound (1.34 g, 99%).

¹H-NMR (DMSO-d₆): δ 12.37 (1H, s); 9.38 (1H, bs); 8.31 (1H, d); 7.93 (1H, d); 7.46-7.52 (2H, m); 7.22-7.41 (3H, m); 7.18 (1H, dd); 6.63-6.76 (3H, m).

### EXAMPLE 20

### 3-(4-Methoxyphenyl)-2-phenyl-1H-pyrrolo[2,3-b]pyridine-5-carbonitrile

The title compound (25 mg, 11%) was synthesized from 6-hydrazino-nicotinonitrile (90 mg, 0.67 mmol), and 1-(4-methoxyphenyl)-2-phenylethanone (152 mg, 0.67 mmol) essentially as described in Example 1 and purified by column chromatography (silica gel; dichloromethane/methanol gradient from 1:0 to 7:3) and crystallized from acetonitrile.

¹H-NMR (DMSO-d₆): δ 12.74 (1H, bs); 8.64 (1H, s); 8.28 (1H, s); 7.50 (2H, d); 7.41-7.34 (3H, m); 7.27 (2H, d); 6.97 (2H, d); 3.18 (3H, s).

¹³C-NMR (DMSO-d₆): δ 158.1; 148.9; 145.7; 136.5; 131.1; 130.7; 130.6; 128.5; 128.4; 124.8; 119.7; 118.7; 114.3; 112.2; 100.3; 55.0.

APCI-MS m/z: 326.4 [MH⁺].

### EXAMPLE 21

### 1-[4-(5-Bromo-2-phenyl-1H-pyrrolo[2,3-b]pyridin-3-yl)phenoxy]-3-[(2-pyrrolidin-1-ylethyl)amino]propan-2-ol dihydrochloride

### a) 4-(5-Bromo-2-phenyl-1H-pyrrolo[2,3-b]pyridin-3-yl)phenol

The title compound (1.05 g, 93%) was synthesized from 5-bromo-3-(4-methoxyphenyl)-2-phenyl-1H-pyrrolo[2,3-b]pyridine (1.17 g, 2.9 mmol) essentially as described in Example 19.

¹H-NMR (DMSO-d₆): δ 12.25 (1H, bs); 9.43 (1H, b); 8.29 (1H, d); 7.89 (1H, d); 7.46-7.51 (2H, m); 7.28-7.39 (3H, m); 7.11 (2H, d); 6.78 (2H, d).

APCI-MS m/z: 365.0/367.0 [MH⁺].

### b) 1-[4-(5-Bromo-2-phenyl-1H-pyrrolo[2,3-b]pyridin-3-yl)phenoxy]-3-[(2-pyrrolidin-1-ylethyl)amino]propan-2-ol dihydrochloride

A mixture of 4-(5-bromo-2-phenyl-1H-pyrrolo[2,3-b]pyridin-3-yl)phenol (174 mg, 0.48 mmol), sodium hydride (60% suspension in mineral oil, 86 mg, 2.14 mmol) and N,N-dimethylformamide (2 ml) was heated at 60° C. for 30 minutes. Epibromohydrin (66 mg, 0.48 mmol) was added and the reaction mixture was further stirred at 60° C. for 1 h. 2-Pyrrolidin-1-ylethanamine (76 mg, 0.68 mmol) was added and reaction was heated at 60° C. for 14 h. Water (1 ml) was added and mixture was eluted through silica gel with dichloromethane/methanol/aqueous ammonia (79.5/20/0.5) and the product was further purified by preparative HPLC (RP-18, acetonitrile/water/trifluoroacetic acid gradient from 10:90:0.1 to 95:5:0.1) to give the title compound (3 mg, 1%). APCI-MS m/z: 535.0/537.0 [MH⁺].

### EXAMPLE 22

### 1-[4-(5-Bromo-2-phenyl-1H-pyrrolo[2,3-b]pyridin-3-yl)phenoxy]-3-pyrrolidin-1-ylpropan-2-ol trifluoroacetate

The title compound (6 mg, 4%) was synthesized from 4-(5-bromo-2-phenyl-1H-pyrrolo[2,3-b]pyridin-3-yl)phenol (82 mg, 0.22 mmol), epibromohydrin and pyrrolidin-3-ol (99 mg, 1.13 mmol) essentially as described in Example 21.

APCI-MS m/z: 508.0/510.1 [MH⁺].

### EXAMPLE 23

### 5-Bromo-3-{4-[2-(1-methylpyrrolidin-2-yl)ethoxy]phenyl}-2-phenyl-1H-pyrrolo[2,3-b]pyridine trifluoroacetate

A mixture of 4-(5-bromo-2-phenyl-1H-pyrrolo[2,3-b]pyridin-3-yl)phenol (100 mg, 0.27 mmol), sodium hydride (60% suspension in mineral oil, 45 mg, 1.25 mmol) and N,N-dimethylformamide (2 ml) was heated at 60° C. for 30 minutes. A mixture of 2-(2-chloroethyl)-1-methylpyrrolidine hydrochloride (51 mg, 0.28 mmol), sodium hydride (60% suspension in mineral oil, 15 mg, 0.42 mmol) and N,N-dimethylformamide (500 μl) was added and the reaction mixture was further stirred at 60° C. for 75 minutes. Water (1 ml) and acetic acid (200 μl, 3.5 mmol) were added and the product was purified by preparative HPLC (RP-18, acetonitrile/water/trifluoroacetic acid gradient from 10:90:0.1 to 95:5:0.1) to give the title compound (73 mg, 45%).

¹H-NMR (CD₃CN): δ 10.37 (sH, s); 8.33 (1H, d); 8.03 (1H, d); 7.46-7.53 (2H, m); 7.37-7.43 (3H, m); 7.23-7.33 (2H, m); 6.94-7.10 (2H, m); 4.68-4.80 (1H, m); 4.10-4.36 (2H, m); 3.32-3.75 (3H, m); 2.85-3.22 (2H, m); 1.80-2.44 (6H, m).

APCI-MS m/z: 476.0/478.0 [MH⁺].

Following the general method of Example 23, the compounds of Examples 24 to 33 were prepared:

### EXAMPLE 24

### 5-Bromo-2-phenyl-3-[4-(2-pyrrolidin-1-ylethoxy)phenyl]-1H-pyrrolo[2,3-b]pyridine trifluoroacetate

The title compound (21 mg, 18%) was synthesized from 4-(5-bromo-2-phenyl-1H-pyrrolo[2,3-b]pyridin-3-yl)phenol (75 mg, 0.21 mmol) and 1-(2-chloroethyl)pyrrolidine hydrochloride (35 mg, 0.21 mmol).

¹H-NMR (CD₃CN): δ 11.12 (1H, s); 8.42 (1H, d); 8.18 (1H, d); 7.41-7.53 (5H, m); 7.10 (2H, d); 6.75 (2H, d); 4.50 (2H, dd); 3.66-3.78 (2H, m); 3.34-3.51 (2H, m); 2.82-2.95 (2H, m); 2.30-2.60 (4H, m).

APCI-MS m/z: 462.1/464.1 [MH⁺].

### EXAMPLE 25

### 5-Bromo-3-[4-(2-morpholin-4-ylethoxy)phenyl]-2-phenyl-1H-pyrrolo[2,3-b]pyridine trifluoroacetate

The title compound (59 mg, 36%) was synthesized from 4-(5-bromo-2-phenyl-1H-pyrrolo[2,3-b]pyridin-3-yl)phenol (100 mg, 0.27 mmol) and 4-(2-chloroethyl)morpholine hydrochloride (53 mg, 0.28 mmol).

¹H-NMR (CD₃CN): δ 8.31 (1H, d); 8.00 (1H, d); 7.28-7.54 (7H, m); 7.01-7.12 (2H, m); 4.36-4.50 (2H, m); 3.84-4.26 (2H, m); 3.52-3.68 (4H, m); 3.20-3.45 (4H, m). APCI-MS m/z: 478.0/480.0 [MH⁺].

### EXAMPLE 26

### 5-Bromo-3-[3-(2-morpholin-4-ylethoxy)phenyl]-2-phenyl-1H-pyrrolo[2,3-b]pyridine trifluoroacetate

The title compound (13.5 mg, 8%) was synthesized from 3-(5-bromo-2-phenyl-1H-pyrrolo[2,3-b]pyridin-3-yl)phenol (101 mg, 0.27 mmol) and 4-(2-chloroethyl)morpholine hydrochloride (51 mg, 0.27 mmol).

APCI-MS m/z: 478.0/480.0 [MH⁺].

### EXAMPLE 27

### 3-[4-(5-Bromo-2-phenyl-1H-pyrrolo[2,3-b]pyridin-3-yl)phenoxy]-N,N-dimethylpropan-1-amine trifluoroacetate

The title compound (37 mg, 34%) was synthesized from 4-(5-bromo-2-phenyl-1H-pyrrolo[2,3-b]pyridin-3-yl)phenol (100 mg, 0.27 mmol) and N-(3-chloropropyl)-N,N-dimethylamine hydrochloride (44 mg, 0.28 mmol).

¹H-NMR (CD₃CN): δ 8.34 (1H, d); 8.03 (1H, d); 7.38-7.44 (5H, m); 7.31 (2H, d); 6.98 (2H, d); 4.17-4.30 (2H, m); 3.43-3.64 (2H, m); 2.64-2.88 (6H, m); 2.34-2.58 (2H, m).

APCI-MS m/z: 450.0/452.0 [MH⁺].

### EXAMPLE 28

### 5-Bromo-3-{4-[2-(2-methoxyethoxy)ethoxy]phenyl}-2-phenyl-1H-pyrrolo[2,3-b]pyridine

The title compound (68 mg, 53%) was synthesized from 4-(5-bromo-2-phenyl-1H-pyrrolo[2,3-b]pyridin-3-yl)phenol (100 mg, 0.27 mmol) and 1-bromo-2-(2-methoxyethoxy)ethane (51 mg, 28 mmol).

¹H-NMR (CD₃Cl: δ 12.08 (1H, s); 8.24 (1H, m); 8.08 (1H, d); 7.56-7.61 (2H, m); 7.38-7.47 (3H, m); 7.32 (2H, d); 6.98 (2H, d); 4.22 (2H, dd); 3.93 (2H, dd); 3.78 (2H, dd); 3.63 (2H, dd); 3.44 (3H, s).

APCI-MS m/z: 466.9/469.0 [MH⁺].

### EXAMPLE 29

### 5-Bromo-3-{3-[2-(1-methylpyrrolidin-2-yl)ethoxy]phenyl}-2-phenyl-1H-pyrrolo[2,3-b]pyridine trifluoroacetate

The title compound (65 mg, 40%) was synthesized from 3-(5-bromo-2-phenyl-1H-pyrrolo[2,3-b]pyridin-3-yl)phenol (101 mg, 0.27 mmol) and 2-(2-chloroethyl)-1-methylpyrrolidine hydrochloride (51 mg, 0.28 mmol).

APCI-MS m/z: 476.0/478.0 [MH⁺].

### EXAMPLE 30

### 3-{4-[3-(Dimethylamino)propoxy]phenyl}-2-phenyl-1H-pyrrolo[2,3-b]pyridine-5-carbonitrile trifluoroacetate

The title compound (34 mg, 60%) was synthesized from 4-(5-cyano-2-phenyl-1H-pyrrolo[2,3-b]pyridin-3-yl)phenol (32 mg, 0.10 mmol) and N-(3-chloropropyl)-N,N-dimethylamine hydrochloride (18 mg, 0.11 mmol).

¹H-NMR (CD₃OD): δ 8.55 (2H, d); 8.19 (1H, d); 7.47-7.53 (2H, m); 7.33-7.38 (3H, m); 7.30 (2H, d); 7.10 (2H, d); 4.27 (2H, dd); 3.73 (2H, dd); 2.96 (6H, s); 2.25 (2H, dddd).

APCI-MS m/z: 397.2 [MH⁺].

### EXAMPLE 31

### 5-{[4(5-Bromo-2-phenyl-1H-pyrrolo[2,3-b]pyridin-3-yl)phenoxy]methyl}-1,3-oxazolidin-2-one trifluoroacetate

The title compound (14 mg, 11%) was synthesized from 4-(5-bromo-2-phenyl-1H-pyrrolo[2,3-b]pyridin-3-yl)phenyl (98 mg, 0.27 mmol) and 5-(chloromethyl)-1,3-oxazolidin-2-one (38 mg, 0.28 mmol).

¹H-NMR (CD₃OD): δ 8.27 (1H, d); 8.18 (1H, d); 7.47-7.53 (2H, m); 7.31-7.40 (3H, m); 6.88-6.99 (4H, m); 4.89-4.97 (1H, m); 4.08 (2H, ddd); 3.69 (1H, dd); 3.50 (1H, dd).

APCI-MS m/z: 365.0/367.0 [MH⁺].

### EXAMPLE 32

### 3-{4-[3-(dimethylamino)propoxy]phenyl}-2-(4-methoxphenyl)-1H-pyrrolo[2,3-b]pyridine-5-carbonitrile trifluoroacetate

The title compound (23 mg, 33%) was synthesized from 3-(4-hydroxyphenyl)-2-(4-methoxyphenyl)-1H-pyrrolo[2,3-b]pyridine-5-carbonitrile (45 mg, 0.13 mmol) and N-(3-chloropropyl)-N,N-dimethylamine hydrochloride (23 mg, 0.15 mmol).

APCI-MS m/z: 427.2 [MH⁺].

### EXAMPLE 33

### (3-{4-[5-Bromo-2-(4-methoxy-phenyl)-1H-pyrrolo[1,3-b]pyridin-3-yl]-phenoxy}-propyl)-dimethylamine

### a) 4-[5-Bromo-2-(4-methoxy-phenyl)-1-H-pyrrolo[2,3-b]pyridin-3-yl]-phenol

(4-Bromo-2-phenyl)-hydrazine (0.5 g, 2.66 mmol) and 2-(4-hydroxy-phenyl)-1-(4 methoxy-phenyl)-ethanone (0.644 g, 2.66 mmol) in benzene (20 mL) containing acetic acid (0.2 mL) was heated at reflux temperature. Water was continuously distilled off using a Dean-Stark trap. After 13 h, the reaction mixture was cooled and triethylamine (0.4 mL) was added. The mixture was evaporated and the residue was re-suspended in water. The precipitate was filtered off to give 4-[2-[(4-bromophenyl)-hydrazono]-2-(4-methoxy-phenyl)-ethyl]-phenol (0.93 g, 84%).

¹H-NMR (DMSO-d₆): δ 10.06 (1H, s), 9.20 (1H, s), 8.18 (1H, d), 7.82 (1H, dd), 7.75 (2H, d), 7.30 (1H, d), 6.97 (2H, d), 6.91 (2H, d), 6.65 (2H, d), 4.16 (2H, s); 3.73 (3H, s).

APCI-MS m/z: 41.1.9; 413.9 [MH⁺].

4-[2-[(4-Bromophenyl)-hydrazono]-2-(4-methoxy-phenyl)-ethyl]-phenol (708 mg, 1.72 mmol) was stirred in an inert atmosphere at 230° C. for 10 minutes. The crude product was purified by column chromatography (silica gel, ethyl acetate-heptane 2:3) and crystallized twice from methanol to give the title compound (23 mg, 3%).

¹H-NMR (DMSO-d₆): δ 12.17 (1H, bs), 9.46 (1H, bs), 8.27 (1H, d), 7.87 (1H, d), 7.44 (2H, d), 7.13 (2H, d), 6.94 (2H, d), 6.81 (2H, d); 3.77 (3H, s).

APCI-MS m/z: 395.0/397.0 [MH⁺]:

### b) (3-{4-[5-Bromo-2-(4-methoxy-phenyl)-1H-pyrrolo[1,3-b]pyridin-3-yl]-phenoxy}-propyl)-dimethyl amine

The title compound (13 mg, 16%) was synthesized from crude 4-[5-bromo-2-(4-methoxy-phenyl)-1-H-pyrrolo[2,3-b]pyridin-3-yl]-phenol (75 mg, purity 87%, 0.14 mmol) and N-(3-chloropropyl)-N,N-dimethylamine hydrochloride.

¹H-NMR (DMSO-d₆): δ 12.22 (1H, bs), 8.28 (1H, d), 7.88 (1H, d), 7.43 (1H, d), 7.23 (1H, d), 6.96 (1H, d), 6.95 (1H, d), 4.02 (2H, t), 3.77 (3H, s), 2.37 (2H, t), 2.15 (6H, s) and 1.86 (2H, p).

APCI-MS m/z: 480.2; 482.1 [MH⁺].

### EXAMPLE 34

### 3-[4-(5-Bromo-2-phenyl-1H-pyrrolo[2,3-b]pyridin-3-yl)phenoxy]propan-1-amine trifluoroacetate

A mixture of 4-(5-bromo-2-phenyl-1H-pyrrolo[2,3-b]pyridin-3-yl)phenol (83 mg, 0.23 mmol), sodium hydride (60% suspension in mineral oil, 22 mg, 0.55 mmol) and N,N-dimethylformamide (2 ml) was heated at 60° C. for 30 minutes. A solution of 2-(3-chloropropyl)-1H-isoindole-1,3 (2H)-dione (61 mg, 0.23 mmol) in N,N-dimethylformamide (500 μl) was added and the reaction mixture was further stirred at 60° C. for 6 h. Water (1 ml) tetrahydrofuran (10 ml), methanol (10 ml) and 2M ethylamine solution in ethanol (4 ml) were added and the reaction mixture was stirred at room temperature for 14 h. The solvents were evaporated off and the residue was dissolved in 1,4-dioxane (10 ml) and water (5 ml) containing sodium hydroxide (4.56 g, 0.11 mol) and the reaction mixture was heated at 100° C. for 2 h. After cooling, the product was extracted into ethyl acetate and purified by preparative HPLC (RP-18; acetonitrile/water/trifluoroacetic acid gradient 10:90:0.1 to 95:5:0.1) to give the title compound (4 mg, 3%).

¹H-NMR (CD₃OD): δ 8.27 (1H, d); 7.96 (1H, d); 7.45-7.53 (3H, m); 7.31-7.38 (2H, d); 7.27 (2H, d); 7.08 (2H, d); 4.17 (2H, dd); 3.19 (2H, dd); 2.14-2.22 (2H, ddd).

APCI-MS m/z: 421.9/423.9 [MH⁺].

### EXAMPLE 35

### 5-Bromo-3-(4-aminomethylphenyl)-2-phenyl-1H-pyrrolo[2,3-b]pyridine

To 4-(5-bromo-2-phenyl-1H-pyrrolo[2,3-b]pyridin-3-yl)benzonitrile (1.00 g, 2.7 mmol) and tetrahydrofuran (5 ml) was added lithium aluminium hydride (1M solution in diethyl ether, 5.7 ml, 5.7 mmol) during 4 h. The reaction mixture was stirred at room temperature for a further 30 minutes and then neutralized by adding methanol and dilute hydrochloric acid. The crude product was purified by column chromatography (silica gel, ethyl acetate/chloroform/methanol/aqueous ammonia gradient 100:0:0:0, 0:95:5:0 and 0:80:19.5:0.5) to give the title compound (0.569 g, 56%).

¹H-NMR (CD₃OD): δ 8.43 (1H, d); 8.23 (1H, d); 7.34-7.55 (9H, m) 4.17 (2H, s).

APCI-MS m/z: 377.0/379.0 [MH⁺].

### EXAMPLE 36

### 5-Bromo-3-[4-(4,5-dihydro-1H-imidazol-2-yl)phenyl]-2-phenyl-1H-pyrrolo[2,3-b]pyridine trifluoroacetate

A mixture of 4-(5-bromo-2-phenyl-1H-pyrrolo[2,3-b]pyridin-3-yl)benzonitrile (82 mg, 0.22 mmol), 1,2-ethanediamine (41 mg, 0.68 mmol), 4-methylbenzenesulfonic acid hydrate (89 mg, 0.47 mmol), glycol (0.3 ml) and DMSO (0.4 ml) was heated at 175° C. for 3 h. After cooling, methanol (1 ml) was added and the product was purified by preparative HPLC (RP-18, acetonitrile/water/trifluoroacetic acid gradient from 10:90:0.1 to 95:5:0.1) to give the title compound (11 mg, 9%).

¹H-NMR (CD₃OD): δ 8.34 (1H, d); 8.14 (1H, d); 7.86 (2H, d); 7.61 (2H, d); 7.46-7.50 (2H, m); 7.36-7.43 (3H, m); 4.22 (4H, s).

APCI-MS m/z: 417.1/419.1 [MH⁺].

### EXAMPLE 37

### 5-Bromo-3-[4-(4,4-dimethyl-4,5-dihydro-1H-imidazol-2-yl)phenyl]-2-phenyl-1H-pyrrolo[2,3-b]pyridine trifluoroacetate

The title compound (5 mg, 4%) was synthesized from 4-(5-bromo-2-phenyl-1H-pyrrolo[2,3-b]pyridin-3-yl)benzonitrile (82 mg, 0.22 mmol) and 2-methylpropane-1,2-diamine (27 mg, 0.31 mmol) essentially as described in Example 36.

¹H-NMR (CD₃OD): δ 8.35 (1H, d); 8.14 (1H, d); 7.85 (2H, d); 7.62 (2H, d); 7.46-7.49 (2H, m); 7.38-7.42 (3H, m); 3.87 (2H, s); 3.35 (2H, s); 1.57 (6H, s).

APCI-MS m/z: 445.0/447.0 [MH⁺].

### EXAMPLE 38

### N-(2-Aminoethyl)-4-(5-bromo-2-phenyl-1H-pyrrolo[2,3-b]pyridin-3-yl)benzamide trifluoroacetate

The title compound (10 mg, 8%) was isolated from the synthesis of 5-bromo-3-[4-(4,5-dihydro-1H-imidazol-2-yl)phenyl]-2-phenyl-1H-pyrrolo[2,3-b]pyridine trifluoroacetate (Example 36).

¹H-NMR (CD₃OD): δ 8.31 (1H, d); 8.07 (1H, d); 7.90 (2H, d); 7.44-7.51 (4H, m); 7.37-7.40 (3H, m); 3.69 (2H, dd); 3.18 (2H, dd).

APCI-MS m/z: 435.1/437.0 [MH⁺].

### EXAMPLE 39

### 3-[[4-(5-Bromo-2-phenyl-1H-pyrrolo[2,3-b]pyridin-3-yl)benzyl](1,2-dihydroxypropyl)amino]propane-1,2-diol trifluoroacetate

To 1-[4-(5-bromo-2-phenyl-1H-pyrrolo[2,3-b]pyridin-3-yl)phenyl]methanamine (58.5 mg, 0.15 mmol) and tetrahydrofuran (5 ml) was added oxiran-2-ylmethanol (77 mg, 5.7 mmol) in three batches during 4 h at 80° C. Methanol (1 ml) was added and the product was purified by preparative HPLC (RP-18, acetonitrile/water/trifluoroacetic acid gradient from 10:90:0.1 to 95:5:0.1) to give the title compound (32 mg, 32%).

¹H-NMR (CD₃OD): δ 8.26 (1H, d); 8.03 (1H, d); 7.40-7.60 (6H, m); 7.30-7.36 (3H, m); 3.88-4.20 (2H, m); 3.48-3.64 (4H, m); 3.20-3.45 (4H, m).

APCI-MS m/z: 526.1/528.1 [MH⁺].

### EXAMPLE 40

### 4-(5-Bromo-2-phenyl-1H-pyrrolo[2,3-b]pyridin-3-yl)benzoic acid

A solution of 4-(5-bromo-2-phenyl-1H-pyrrolo[2,3-b]pyridin-3-yl)benzonitrile (135 mg, 0.36 mmol), conc. sulphuric acid (2 ml), water (2 ml) and 1,4-dioxane (2 ml) was heated at 120° C. for 2 h. After cooling, water (100 ml) was added and the precipitate was purified by preparative HPLC (RP-18, acetonitrile/water/trifluoroacetic acid gradient from 10:90:0.1 to 95:5:0.1) to give the title compound (33 mg, 23%).

¹H-NMR (CD₃OD): δ 8.60 (1H, d); 8.05 (1H, d); 7.35-7.54 (9H, m).

APCI-MS m/z: 393.0/395.0 [MH⁺].

### EXAMPLE 41

### N⁵-[4-(5-Bromo-2-phenyl-1H-pyrrolo[2,3-b]pyridin-3-yl)benzyl]glutamine trifluoroacetate

A mixture of Boc-Glu-OtBu (53 mg, 0.18 mmol), HATU (72 mg, 0.19 mmol) and dichloromethane (2 ml) was adjusted to pH 8 with diisopropylethyl amine. After 20 minutes a solution of 1-[4-(5-bromo-2-phenyl-1H-pyrrolo[2,3-b]pyridin-3-yl)phenyl]methanamine (65 mg, 0.17 mmol) in NMP (1 ml) was added and the pH was adjusted to 8 with diisopropylethyl amine. After 6 h, trifluoroacetic acid (1.5 ml) was added. After 17 h, the solvents were evaporated off and the crude product was purified by preparative HPLC (RP-18, acetonitrile/water/trifluoroacetic acid gradient from 10:90:0.1 to 95:5:0.1) to give the title compound (41 mg, 38%).

¹H-NMR (CD₃OD): δ 8.40 (1H, d); 8.05 (1H, d); 7.37-7.61 (9H, m); 4.17 (2H, dd); 2.26-2.60 (4H, m).

APCI-MS m/z: 507.0/509.0 [MH⁺].

### EXAMPLE 42

### 3-(4-Hydroxyphenyl)-2-phenyl-1H-pyrrolo[2,3-b]pyridine-5-carbonitrile

A mixture of 4-(5-bromo-2-phenyl-1H-pyrrolo[2,3-b]pyridin-3-yl)phenol (355 mg, 0.97 mmol), zinc cyanide (137 mg, 1.17 mmol), tris(dibenzylideneacetone)dipalladium(0) (89 mg, 97 μmol), bis(diphenylphosphine)ferrocene (129 mg, 0.23 mmol) and N,N-dimethylformamide (10 ml) was stirred at 130° C. for 20 h. Ethyl acetate (100 ml) was added and the organic phase was washed with water (2×50 ml), dried and concentrated in vacuo. The crude product was purified by column chromatography (silica gel, ethyl acetate/heptane gradient from 3:7 to 8:2) and crystallization (6 ml tetrahydrofuran/heptane 5:1) to give the title compound (115 mg, 38%).

¹H-NMR (DMSO-d₆): δ 12.65 (1H, s); 9.50 (1H, s); 8.62 (1H, d); 8.26 (1H, d); 7.50 (2H, dd); 7.32-7.43 (3H, m); 7.15 (2H, d); 6.79 (2H, d).

APCI-MS m/z: 312.1 [MH⁺].

Following the general method of Example 42, the compounds of Examples 43 to 45 were prepared:

### EXAMPLE 43

### 3-[4-(Aminomethyl)phenyl]-2-phenyl-1H-pyrrolo[2,3-pyridine-5-carbonitrile trifluoroacetate

The title compound (12 mg, 10%) was synthesized from 1-[4-(5-bromo-2-phenyl-1H-pyrrolo[2,3-b]pyridin-3-yl)phenyl]methanamine (104 mg, 0.27 mmol) and purified by preparative HPLC (RP-18, acetonitrile/water/trifluoroacetic acid gradient from 10:90:0.1 is to 95:5:0.1).

¹H-NMR (CD₃OD): δ 8.58 (1H, d); 8.24 (1H, d); 7.45-7.53 (6H, m); 7.32-7.40 (3H, m); 4.18 (2H, s)

APCI-MS m/z: 325.4 [MH⁺, weak], 308.1 (MH⁺ —NH₃].

### EXAMPLE 44

### 3-(4-Morpholin-4-ylphenyl)-2-phenyl-1H-pyrrolo[2,3-b]pyridine-5-carbonitrile

The title compound (5 mg, 11%) was synthesized from 5-bromo-3-(4-morpholin-4-ylphenyl)-2-phenyl-1H-pyrrolo[2,3-b]pyridine (50 mg, 0.115 mmol).

¹H-NMR (DMSO-d6): δ 12.61 (1H, s); 8.58 (1H, d); 8.21 (1H, d); 7.28-7.44 (7H, m); 6.93 (2H, d); 3.71 (4H, dd); 3.16 (4H, dd).

APCI-MS m/z: 381.2 [MH⁺].

### EXAMPLE 45

### 3-(4-Hydroxyphenyl)-2-(4-methoxyphenyl)-1H-pyrrolo[2,3-b]pyridine-5-carbonitrile

The title compound (50 mg, 93%) was synthesized from 4-[5-bromo-2-(4-methoxyphenyl)-1H-pyrrolo[2,3-b]pyridin-3-yl]phenol (62 mg, 0.16 mmol).

¹H-NMR (DMSO-d₆): δ 12.59 (1H, s); 9.48 (1H, s); 8.48 (1H, d); 8.20 (1H, d); 7.44 (2H, d); 7.15 (2H, d); 6.95 (2H, d); 6.80 (2H, d).

APCI-MS m/z: 342.1 [MH⁺].

### EXAMPLE 46

### 5-Bromo-2-phenyl-3-pyrrol-1-yl-1H-pyrrolo[2,3-b]pyridine

5-Bromo-2-phenyl-1H-pyrrolo[2,3-b]pyridin-3-ylamine (60 mg, 0.2 mmol) was suspended in acetic acid (20 ml) and 2,5-dimethoxytetrahydrofuran (0.030 ml, 0.22 mmol) added. The mixture was refluxed for 1.5 h, cooled and concentrated in vacuo. The residue was chromatographed (silica gel, heptane/ethyl acetate gradient from 1:0 to 0:1). This gave the to product as an off-white powder (59.1 mg, 84%). An analytical sample was further purified by recrystallisation from acetonitrile/toluene/ethyl acetate.

¹H-NMR (DMSO-d₆): δ 12.59 (1H, bs); 8.37 (1H, s); 7.80 (1H, s); 7.40-7.25 (5H, m); 6.86 (2H, t); 6.29 (2H, t).

APCI-MS m/z: 338.0/340.0 [MH⁺].

### EXAMPLE 47

### 5-Cyano-2-(4-methoxy-phenyl)-3-pyrrol-1-yl-1H-pyrrolo[2,3-b]pyridine

The title compound (25 mg, 16%) was synthesized from 6-hydrazino-nicotinonitrile (70 mg, 0.5 mmol), and 1-(4-methoxyphenyl)-2-(1H-pyrrol-1-yl)ethanone (110 mg, 0.5 mmol) essentially as described for Example 1 and purified by preparative HPLC (RP-18, acetonitrile/water/trifluoroacetic acid gradient from 10:90:0.1 to 95:5:0.1).

¹H-NMR (CDCl₃): δ 11.18 (1H, bs); 8.53 (1H, s); 8.17 (1H, s); 7.25 (2H, d); 6.94 (2H; d); 6.76 (2H, t); 6.41 (2H, t); 3.84 (3H, s).

APCI-MS m/z: 315.1 [MH⁺].

### EXAMPLE 48

### 5-Bromo-3-(2,5-dimethyl-pyrrol-1-yl)-2-phenyl-1H-pyrrolo[2,3-b]pyridine

5-Bromo-2-phenyl-1H-pyrrolo[2,3-b]pyridin-3-ylamine (48 mg, 0.16 mmol) was suspended in acetic acid (20 ml) and hexane-2,5-dione (0.025 ml, 0.20 mmol) added. The mixture was refluxed for 1.5 h and then concentrated in vacuo. The crude product was purified by preparative HPLC (RP-18, acetonitrile/water/trifluoroacetic acid gradient from 10:90:0.1 to 95:5:0.1) to give the title compound (17 mg, 29%).

¹H-NMR (DMSO-d₆): δ 11.55 (1H, bs); 8.36 (1H, s); 7.77 (1H, s); 7.39-7.31 (5H, m); 5.93 (2H, s); 1.86 (6H, s).

APCI-MS m/z: 366.3/368.3 [MH⁺].

### EXAMPLE 49

### {3-[4-(5-Methyl-3-pyrimidin-5-yl-1H-pyrrolo[2,3-b]pyridin-2-yl)phenoxy]propyl}dimethylamine

2-Iodo-5-methyl-3-pyrimidin-5-yl-1H-pyrrolo[2,3-b]pyridine (0.31 g, 0.93 mmol), {4-[3-(dimethylamino)propoxy]phenyl}boronic acid (prepared as Example 70a, 0.23 g, 1.03 mmol), potassium carbonate (0.35 g, 2.53 mmol) and 1,1′ bis(diphenylphosphino)ferrocenedichloropalladium(II) (98 mg, 0.12 mmol) were suspended in dioxane (10 ml). The mixture was degassed with argon and stirred at 100° C. for 15 h. Reaction mixture was acidified with aqueous HCl and partitioned between ethyl acetate and water. The aqueous layer was collected, basified with sodium bicarbonate and extracted with ethyl acetate. The organic layer was evaporated and purified by preparative HPLC (RP-18, acetonitrile/water/acetic acid gradient from 10:90:0.1 to 95:5:0.1). The acetonitrile was evaporated, the remaining solution basified with sodium bicarbonate and extracted with ethyl acetate. The organic layer was dried and evaporated to give the title compound (218 mg, 55%).

¹H-NMR (400 MHz, CDCl₃): δ 11.21 (s, 1H), 9.20 (s, 1H), 8.85 (s, 2H), 8.13 (d, J 1.6 Hz, 1H), 7.80 (d, J 1.1 Hz, 1H), 7.45 (d, J 8.7 Hz, 2H), 6.99 (d, J 8.7 Hz, 2H), 4.12 (t, J 8.0 Hz, 2H), 2.71 (t, J 7.4 Hz, 2H), 2.48 (s, 3H), 2.46 (s, 6H), 2.19-2.09 (m, 2H). APCI-MS m/z: 388.1 [MH⁺].

### a) 2-Iodo-5-methyl-3-pyrimidin-5-yl-1H-pyrrolo[2,3-b]pyridine

A mixture of 5-methyl-3-pyrimidin-5-yl-2-(trimethylsilyl)-1H-pyrrolo[2,3-b]pyridine (0.14 g, 0.57 mmol), N-iodosuccinimide (0.23 g, 1.02 mmol) and 1,2-dichloroethane (3 ml) was heated in microwave reactor at 90° C. for 20 min. This reaction was performed six times and the reaction mixtures were combined, poured into aqueous sodium thiosulfate, filtered and washed with ethanol to yield the subtitle compound (1.03 g, 99%).

¹H-NMR (400 MHz, CDCl₃): δ 9.08 (s, 1H), 8.89 (s, 2H), 8.36 (s, 1H), 7.59 (s, 1H), 2.34 (s, 3H).

APCI-MS m/z: 336.9 [MH+].

### b) 5-Methyl-3-pyrimidin-5-yl-2-(trimethylsilyl)-1H-pyrrolo[2,3-b]pyridine

A mixture of 3-iodo-5-methylpyridin-2-amine (2.0 g, 8.64 mmol), bis(triphenylphosphine)palladium(II) chloride (0.48 g, 0.58 mmol), 1,4-diazabicyclo(2,2,2)octane (1.66 g, 14.8 mmol), 5-[(trimethylsilyl)ethynyl]pyrimidine (1.97 g, 11.2 mmol) and N,N-dimethylformamide (10 ml) was heated to 110° C. for 16 h. The reaction mixture was evaporated and the crude product was purified by column chromatography (silica gel, ethyl acetate-heptane gradient from 0:100 to 100:0) to yield the subtitle compound (0.86 g, 35%).

¹H-NMR (400 MHz, DMSO-d₆): δ 11.75 (s, 1H), 9.40 (s, 1H), 8.83 (s, 2H), 8.16 (d, J 1.7 Hz, 1H), 7.64-7.62 (m, 1H), 2.34 (s, 3H), 0.44 (s, 9H).

APCI-MS m/z: 283.2 [MH⁺].

### c) 3-Iodo-5-methylpyridin-2-amine

Trifluoromethanesulfonic acid (10 ml) was added under stirring to 2-amino-5-methylpyridine (5.2 g, 0.048 mol). To this mixture solid N-iodo-succinimide (16 g, 0.071 mol) was added portionwise during 5 min. Stirring was continued for an additional 10 min. and the reaction mixture was poured into aqueous sodium bicarbonate. An excess of sodium thiosulfate was added and the slurry was extracted twice with ethyl acetate. The combined organic layers were washed with aqueous Na₂S₂O₂, brine and then dried over sodium sulfate. Filtration through a plug of silica gel yielded after evaporation the subtitle compound (6.8 g, 60%).

¹H-NMR (300 MHz, CDCl₃) δ 7.87-7.83 (m, 1H), 7.72 (M, 1H), 4.76 (s, 2H), 2.52 (s, 3H).

APCI-MS m/z: 235.0 [MH⁺].

### EXAMPLE 50

### {3-[4-(5-Fluoro-3-pyrimidin-5-yl-1H-pyrrolo[2,3-b]pyridin-2-yl)phenoxy]propyl}dimethylamine

The title compound (46 mg, 37%) was synthesized from 2-iodo-5-fluoro-3-pyrimidin-5-yl-1H-pyrrolo[2,3-b]pyridine (0.11 g, 0.32 mmol) by the procedure of Example 49.

¹H-NMR (400 MHz, DMSO-d₆): δ 12.49 (s, 1H), 8.86 (s, 1H), 8.74 (s, 2H), 8.27 (t, J 2.1 Hz, 1H), 7.88 (q, J 4.1 Hz, 1H), 7.37 (d, J 8.9 Hz, 2H), 6.98 (d, J 9.2 Hz, 2H), 4.02 (t, 16.5 Hz, 2H), 2.33 (t, J 7.2 Hz, 2H), 2.13 (s, 6H), 1.83 (quintet, J 6.7 Hz, 2H).

APCI-MS m/z: 392.2 [MH⁺].

### EXAMPLE 51

### 2-{4-[3-(dimethylamino)propoxy]phenyl}-4-methyl-3-pyridin-3-yl-1H-pyrrolo [2,3-b]pyridine-5-carbonitrile

The title compound (2.2 mg, 1.6%) was synthesized from 2-iodo-4-methyl-3-pyridin-3-yl-1H-pyrrolo[2,3-b]pyridine-5-carbonitrile (0.12 g, 0.32 mmol) by the procedure of Example 49.

¹H-NMR (400 MHz, CDCl₃): δ 12.85 (s, 1H), 8.69 (q, J 2.1 Hz, 1H), 8.62 (d, J 1.9 Hz, 1H), 8.47 (s, 1H), 7.73 (d, J 7.8 Hz, 1H), 7.41 (t, J 6.2 Hz, 1H), 7.26 (d, J 9.0 Hz, 2H), 6.87 (d, J 9.0 Hz, 2H), 4.14 (t, J 5.6 Hz, 2H), 3.30-3.22 (m, 2H), 2.88 (s, 6H), 2.49-2.42 (m, 2H); 2.40 (s, 3H).

APCI-MS m/z: 412.1 [MH⁺].

### EXAMPLE 52

### 5-Chloro-2-[5-(piperazin-1-ylcarbonyl)-1H-pyrrol-3-yl]-3-pyridin-3-yl-1H-pyrrolo[2,3-b]pyridine bis(trifluoroacetate)

The title compound (12 mg, 6.2%) was synthesized from 5-chloro-2-iodo-3-pyridin-3-yl-1H-pyrrolo[2,3-b]pyridine (0.122 g, 0.300 mmol) and tert-butyl 4-{[4-(4,4,5,5-tetramethyl-1,3,2-dioxaborolan-2-yl)-1H-pyrrol-2-yl]carbonyl}piperazine-1-carboxylate (0.123 g, 0.30 mmol) by the procedure of Example 49.

¹H-NMR (400 MHz, CD₃OD): δ 8.73 (s, 1H), 8.60 (d, J 5.3 Hz, 1H), 8.23 (t, J 3.9 Hz, 1H), 8.19 (d, J 2.1 Hz, 1H), 7.89 (d, J 2.2 Hz, 1H), 7.76-7.71 (m, 1H), 7.20 (d, J 1.6 Hz, 1H), 6.70 (d, J 1.5 Hz, 1H), 3.98 (t, J 5.4 Hz, 4H), 3.29-3.27 (m, 4H).

APCI-MS m/z: 407.0 [MH⁺].

### EXAMPLE 53

### 5-Chloro-2-[5-(piperazin-1-ylcarbonyl)-1H-pyrrol-3-yl]-3-pyrimidin-5-yl-1H-pyrrolo[2,3-b]pyridine bis(trifluoroacetate)

The title compound (1.1 mg, 1.1%) was synthesized from 5-chloro-2-iodo-3-pyrimidin-5-yl-1H-pyrrolo[2,3-b]pyridine (0.60 g, 0.17 mmol) and tert-butyl 4-([4-(4,4,5,5-tetramethyl-1,3,2-dioxaborolan-2-yl)-1H-pyrrol-2-yl]carbonyl}piperazine-1-carboxylate (0.250 g, 0.62 mmol) by the procedure of Example 49.

¹H-NMR (400 MHz, CD₃OD): δ 9.12 (s, 1H), 9.12 (s, 2H), 8.20 (d, J 2.1 Hz, 1H), 7.94 (d, J 2.1 Hz, 1H), 7.25-7.23 (m, 1H), 6.67-6.66 (m, 1H), 3.97 (t, J 5.4 Hz, 4H), 3.29-3.26 (m, 4H).

APCI-MS m/z: 408.0 [MH⁺].

### EXAMPLE 54

### {3-[4-(4,5-Dichloro-3-pyrimidin-5-yl-1H-pyrrolo[2,3-b]pyridin-2-yl)phenoxy]propyl}dimethylamine

The title compound (34 mg, 16%) was synthesized from 4,5-dichloro-2-iodo-3-pyrimidin-5-yl-1H-pyrrolo[2,3-b]pyridine (0.19 g, 0.49 mmol) by the procedure of Example 49.

¹H-NMR (400 MHz, CDCl₃): δ12.48 (s, 1H), 9.22 (s, 1H), 9.22 (s, 1H), 8.20 (s, 1H), 7.30 (d, J 7.9 Hz, 2H), 6.95 (d, J 7.9 Hz, 2H), 4.10-4.02 (m, 2H), 2.56-2.48 (m, 2H), 2.31 (s, 6H), 2.06-1.95 (m, 2H).

APCI-MS m/z: 442.3 [MH⁺].

### EXAMPLE 55

### {3-[4-(5-Bromo-4-methyl-3-pyridin-3-yl-1H-pyrrolo[2,3-b]pyridin-2-yl)phenoxy]propyl}dimethylamine

The title compound (33 mg, 27%) was synthesized from 5-bromo-2-iodo-4-methyl-3-pyridin-3-yl-1H-pyrrolo[2,3-b]pyridine (0.11 g, 0.26 mmol) by the procedure of Example 49.

¹H-NMR (400 MHz, CDCl₃): δ 12.28 (s, 1H), 8.67-8.63 (m, 2H), 8.26 (s, 1H), 7.69 (d, J 8.1 Hz, 1H), 7.35 (q, J 4.2 Hz, 1H), 7.30 (d, J 8.3 Hz, 2H), 6.90 (d, J 8.9 Hz, 2H), 4.06 (t, J 6.4 Hz, 2H), 2.52 (t, J 7.3 Hz, 2H), 2.31 (s, 6H), 2.26 (s, 3H), 2.01 (quintet, J 6.9 Hz, 2H).

APCI-MS m/z: 465.3 [MH⁺].

### EXAMPLE 56

### 5-Chloro-3-pyridin-3-yl-2-(1H-pyrrol-2-yl)-1H-pyrrolo[2,3-b]pyridine

The title compound (7 mg, 15%) was synthesized from 5-chloro-2-iodo-3-pyridin-3-yl-1H-pyrrolo[2,3-b]pyridine (58 mg, 0.16 mmol) and 1-(tert-butylcarbonyl)pyrrole-2-boronic acid (41 mg, 0.19 mmol) by the procedure of Example 49.

¹H-NMR (300 MHz, CDCl₃): δ 10.15 (s, 1H), 8.50 (d, J 2.0 Hz, 1H), 8.37 (d, J 5.4 Hz, 1H), 8.00-7.91 (m, 2H), 7.60 (d, J 2.3 Hz, 1H), 7.49 (q, J 4.4 Hz, 1H), 6.70-6.65 (m, 1H), 6.15-6.11 (m, 1H), 6.02-5.98 (m, 1H).

APCI-MS m/z: 295.0 [MH⁺].

### EXAMPLE 57

### 5-Chloro-3-pyridin-3-yl-2-(1H-pyrrol-3-yl)-1H-pyrrolo[2,3-b]pyridine trifluoroacetate

The title compound (4 mg, 3%) was synthesized from 5-chloro-2-iodo-3-pyridin-3-yl-1H-pyrrolo[2,3-b]pyridine (103 mg, 0.29 mmol) and 1-(trisopropylpyl)pyrrole-3-boronic acid (95 mg, 0.36 mmol) by the procedure of Example 49.

¹H-NMR (300 MHz, CD₃OD): δ 8.90 (d, J 1.9 Hz, 1H), 8.74 (d, J 5.5 Hz, 1H), 8.58 (d, J 8.3 Hz, 1H), 8.30 (d, J 2.4 Hz, 1H), 8.14 (d, J 2.3 Hz, 1H), 8.04 (q, J 4.8 Hz, 1H), 7.72 (d, J 2.8 Hz, 1H), 7.58 (q, J 4.1 Hz, 1H), 6.56 (d, J 9.6 Hz, 1H).

APCI-MS m/z: 295.0 [MH+].

### EXAMPLE 58

### 5-Chloro-4-methoxy-3-pyridin-3-yl-2-(1H-pyrrol-3-yl)-1H-pyrrolo[2,3-b]pyridine trifluoroacetate

The title compound (3 mg, 6%) was synthesized from 5-chloro-3-iodo-4-methoxypyridin-2-amine (43 mg, 0.11 mmol) and 1-(trisopropylpyl)pyrrole-3-boronic acid (301 mg, 1.12 mmol) by the procedure of Example 49.

¹H-NMR (400 MHz, CD₃OD): δ 10.79 (s, 1H), 8.93 (s, 1H), 8.69 (d, J 5.6 Hz, 1H), 8.55 (d, J 8.2 Hz, 1H), 8.15 (s, 1H), 7.94 (q, J 4.6 Hz, 1H), 6.99 (s, 1H), 6.80-6.77 (m, 1H), 6.13-6.10 (m, 1H), 3.55 (s, 3H).

APCI-MS m/z: 325.2 [MH⁺].

### EXAMPLE 59

### 5-Chloro-2-(6-chloropyridin-3-yl)-3-pyrimidin-5-yl-1H-pyrrolo[2,3-b]pyridine

2-Iodo-5-chloro-3-pyrimidin-5-yl-1H-pyrrolo[2,3-b]pyridine (prepared as Example 49a, 1.10 g, 3.09 mmol), 2-chloropyridine-5-boronic acid (583 mg, 3.70 mmol), potassium carbonate (1.28 g, 9.27 mmol) and 1,1′-bis(diphenylphosphino)ferrocenedichloro-palladium(II) (126 mg, 0.15 mmol) were suspended in dioxane (10 ml). The mixture was degassed with argon and stirred at 100° C. for 2 h. The reaction mixture was evaporated and the crude product was purified by column chromatography (silica gel, ethyl acetate-heptanes gradient from 0:100 to 100:0) to give the title compound (1.01 g, 96%).

¹H-NMR (300 MHz, CDCl₃): δ 11.93 (s, 1H), 9.24 (s, 1H), 9.24 (s, 2H), 8.64 (d, J 2.3 Hz, 1H), 8.26 (d, J 2.3 Hz, 1H), 7.99 (d, J 2.3 Hz, 1H), 7.74 (q, J 3.7 Hz, 1H), 7.45 (d, J 8.4 Hz, 1H).

APCI-MS m/z: 342.1 [MH⁺].

### EXAMPLE 60

### (2-{[5-(5-Chloro-3-pyrimidin-5-yl-1H-pyrrolo[2,3-b]pyridin-2-yl)pyridin-2-yl]oxy}ethyl)methylamine tris(trifluoroacetate)

A mixture of 5-chloro-2-(6-chloropyridin-3-yl)-3-pyrimidin-5-yl-1H-pyrrolo[2,3-b]pyridine (Example 59, 37 mg, 0.11 mmol) potassium tert-butoxide (42 mg, 0.37 mmol), 2-(methylamino)ethanol (23 mg, 0.31 mmol) and DMF (500 μl) was heated to 100° C. for 2 h. After evaporation of the volatiles the crude product was purified by preparative HPLC (X-Terra RP-18, acetonitrile/water/NH₄OH gradient from 10:90:0.2 to 95:5:0.2) to yield the title compound (8 mg, 10%).

¹H-NMR (400 MHz, CDCl₃): δ 8.93 (s, 1H), 8.93 (s, 2H), 8.08 (q, J 5.4 Hz, 1H), 7.75 (d, J 2.8 Hz, 1H), 7.57 (q, J 3.3 Hz, 1H), 6.73 (d, J 9.3 Hz, 1H), 4.45 (t, J 4.9 Hz, 2H), 3.22 (t, J 4.7 Hz, 2H), 2.58 (s, 3H).

APCI-MS m/z: 381.3 [MH⁺].

### EXAMPLE 61

### N-[5-(5-Chloro-3-pyrimidin-5-yl-1H-pyrrolo[2,3-b]pyridin-2-yl]-N,N′,N′-trimethylpropane-1,3-diamine trihydrochloride

A mixture of 5-chloro-2-(6-chloropyridin-3-yl)-3-pyrimidin-5-yl-1H-pyrrolo[2,3-b]pyridine (Example 59, 22 mg, 0.07 mmol) and N,N,N′-trimethylpropane-1,3-diamine (300 μl) was heated to 160° C. for 1 h. The crude product was purified by column chromatography (silica gel, chloroform-methanol-NH₄OH gradient from 100:0:0 to 70:29:1) and converted into the HCl salt to yield the title compound (22 mg, 59%).

¹H-NMR (400 MHz, DMSO-d₆): δ 12.62 (s, 1H), 9.12 (s, 1H), 8.81 (s, 2H), 8.28 (d, J 2.4 Hz, 1H), 8.18 (d, J 2.4 Hz, 1H), 8.05 (d, J 2.4 Hz, 1H), 7.62 (d, J 9.4 Hz, 1H), 6.80 (d, J 9.2 Hz, 1H), 3.05 (s, 3H), 2.78 (d, J 4.9 Hz, 2H), 2.75-2.71 (m, 6H), 1.97-1.87 (m, 2H).

APCI-MS m/z: 422.9 [MH⁺].

### EXAMPLE 62

### N′-[5-(5-chloro-3-pyrimidin-5-yl-1H-pyrrolo[2,3-b]pyridin-2-yl)pyridin-2-yl]-N,N-dimethylpropane-1,3-diamine tris(trifluoroacetate)

The title compound (15 mg, 29%) was synthesised from 5-chloro-2-(6-chloropyridin-3-yl)-3-pyrimidin-5-yl-1H-pyrrolo[2,3-b]pyridine (Example 59, 22 mg, 0.07 mmol) and N,N-dimethylpropane-1,3-diamine (300 μl) as described in Example 61.

¹H-NMR (400 MHz, DMSO-d₆): δ 12.56 (s, 1H), 9.37 (s, 1H), 8.80 (s, 2H), 8.27 (d, J 2.4 Hz, 1H), 8.10 (d, J 3.0 Hz, 1H), 8.05 (d, J 2.6 Hz, 1H), 7.49 (d, J 9.4 Hz, 1H), 6.57 (d, J 9.4 Hz, 1H), 3.37-3.30 (m, 2H), 3.13-3.06 (m, 2H), 2.76 (s, 6H), 1.87 (quintet, J 7.1 Hz, 2H).

### EXAMPLE 63

### N-{3-[4-(5-chloro-3-pyrimidin-5-yl-1H-pyrrolo[2,3-b]pyridin-2-yl)phenoxy]propyl}-N,N-dimethylamine

The title compound (20 mg, 25%) was prepared from 5-chloro-2-iodo-3-pyrimidin-5-yl-1H-pyrrolo[2,3-b]pyridine (70 mg, 196 μmol) by the procedure of Example 49.

¹H-NMR (400 MHz, DMSO-d₆): δ 12.59 (s, 1H), 9.11 (s, 1H), 8.76 (s, 2H), 8.29 (d, J 2.3 Hz, 1H), 8.06 (d, J 2.3 Hz, 1H), 7.39 (d, J 8.6 Hz, 2H), 6.99 (d, J 8.7 Hz, 2H), 4.03 (t, J 6.3 Hz, 2H), 2.35 (t, J 7.1 Hz, 2H), 2.14 (s, 6H), 1.85 (q, J 6.7 Hz, 2H).

APCI-MS m/z: 408.3 [MH⁺].

### EXAMPLE 64

### {3-[4-(5-Chloro-4-methoxy-3-pyridin-3-yl-1H-pyrrolo[2,3-b]pyridin-2-yl)phenoxy]propyl}dimethylamine

The title compound (5 mg, 7%) was prepared from 5-chloro-2-iodo-4-methoxy-3-pyridin-3-yl-1H-pyrrolo[2,3-b]pyridine (67 mg, 174 μmol) by the procedure of Example 49.

¹H-NMR (400 MHz, DMSO-d₆): δ 12.70 (s, 1H), 8.49 (m, 1H), 8.47 (s, 1H), 8.21 (s, 1H), 7.76 (d, J 7.5 Hz, 1H), 7.39 (m, 1H), 7.29 (d, J 8.6 Hz, 2H), 6.90 (d, J 8.7 Hz, 2H), 3.99 (t, J 6.4 Hz, 2H), 2.33 (t, J 7.2 Hz, 2H), 2.13 (s, 6H), 1.83 (q, J 6.5 Hz, 2H).

APCI-MS m/z: 437.1 [MH⁺].

### EXAMPLE 65

### N-(2-{[5-(5-chloro-3-pyrimidin-5-yl-1H-pyrrolo[2,3-b]pyridin-2-yl)pyridin-2-yl]oxy}ethyl)urea bis(trifluoroacetate)

Sodium hydride (50% in mineral oil, 30 mg, 614 [mol) was dissolved in dry DMF (5 ml). 2-Hydroxyethylurea (11 mg, 105 μmol) was added and the solution stirred for 5 min before 5-chloro-2-(6-chloropyridin-3-yl)-3-pyrimidin-5-yl-1H-pyrrolo[2,3-b]pyridine (Example 59, 30 mg, 88.0 μmol) was added. The mixture was first heated to 80° C. for 30 min and then to 100° C. for 2 h. The crude product was purified by preparative HPLC (acetonitrile/water/TFA gradient from 10:90:0.1 to 95:5:0.1) to give the title compound (1 mg, 3%).

¹H-NMR (300 MHz, DMSO-d₆): δ 12.74 (s, 1H), 9.14 (s, 1H), 8.81 (s, 2H), 8.33 (d, J 2.4 Hz, 1H), 8.30 (d, J 2.6 Hz, 1H), 8.12 (d, J 2.4 Hz, 1H), 7.75 (dd, J 8.6, 2.6 Hz, 1H), 6.90 (d, J 9.2 Hz, 1H), 6.20 (s, 1H), 5.58 (s, 1H), 4.26 (t, J 5.9 Hz, 2H), 3.24 (m, 2H).

APCI-MS m/z: 410.1 [MH⁺].

### EXAMPLE 66

### 2-{[5-(5-Chloro-3-pyrimidin-5-yl-1H-pyrrolo[2,3-b]pyridin-2-yl)pyridin-2-yl]oxy}ethanol bis(trifluoroacetate)

A mixture of 5-chloro-2-(6-chloropyridin-3-yl)-3-pyrimidin-5-yl-1H-pyrrolo[2,3-b]pyridine (Example 59, 15 mg, 44.0 μmol), ethyleneglycol (245 μl, 4.38 mmol) and potassium tert-butoxide (25 mg, 219 μmol) and dioxane (1 ml) was reacted in a microwave reactor at 150° C. for 7 min. The reaction mixture was diluted with water and extracted with dichloromethane. The combined organic layers were dried over sodium sulfate, filtered and concentrated in vacuo. The crude product was purified by preparative HPLC (RP-18, acetonitrile/water/TFA gradient from 10:90:0.1 to 95:5:0.1) to give the title compound (6 mg, 37%).

¹H-NMR (400 MHz, DMSO-d₆): δ 12.76 (s, 1H), 9.16 (s, 1H), 8.81 (s, 2H), 8.33 (d, J 2.2 Hz, 1H), 8.28 (d, J 2.5 Hz, 1H), 8.12 (d, J 2.2 Hz, 1H), 6.90 (d, J 8.7 Hz, 1H), 7.75 (dd, J 8.7, 2.5 Hz, 1H), 4.30 (t, J 5.1 Hz, 2H), 3.71 (t, J 5.1 Hz, 2H).

APCI-MS m/z: 368.0 [MH⁺].

### EXAMPLE 67

### 2-[6-(4-Acetylpiperazin-1-yl)pyridin-3-yl]-5-chloro-3-pyrimidin-5-yl-1H-pyrrolo[2,3-b]pyridine bis(trifluoroacetate)

The title compound (1 mg, 5%) was synthesized from 5-chloro-2-(6-chloropyridin-3-yl)-3-pyrimidin-5-yl-1H-pyrrolo[2,3-b]pyridine (Example 59, 15 mg, 44.0 μmol) and 4-acetylpiperazine (515 μl, 4.38 mmol) by the procedure of Example 66.

¹H-NMR (400 MHz, DMSO-d₆): δ 12.65 (s, 1H), 9.15 (s, 1H), 8.83 (s, 2H), 8.29 (d, J 2.2 Hz, 1H), 8.24 (d, J 2.4 Hz, 1H), 8.06 (d, J 2.2 Hz, 1H), 7.62 (dd, J 8.9, 2.4 Hz, 1H), 6.94 (d, J 9.0 Hz, 1H), 3.63 (d, J 5.2 Hz, 2H), 3.56 (s, 6H), 2.05 (s, 3H).

APCI-MS m/z: 434.1 [MH⁺].

### EXAMPLE 68

### 5-Chloro-3-(4,5-dihydropyrimidin-5-yl)-2-[6-(4-methylpiperazin-1-yl)pyridin-3-yl]-1H-pyrrolo[2,3-b]pyridine

5-Chloro-2-iodo-3-pyrimidin-5-yl-1H-pyrrolo[2,3-b]pyridine (prepared by analogy to Example 49a, 15 mg, 44.0 μmol), 4-methylpiperazine (490 μl, 4.38 mmol), potassium tert-butoxide (8 mg, 66.0 μmol), 1,3-di-i-propylimidazolium chloride (0.5 mg, 2.00 μmol) and Pd₂(dba)₃ (1 mg, 0.90 μmol) were suspended in dioxane (1 ml). The mixture was degassed with argon and stirred at 95° C. for 4 days. The reaction mixture was poured into brine and extracted with ether. The combined organic layers were dried with sodium sulfate, filtered and concentrated. The crude product was purified twice by preparative HPLC (acetonitrile/water/TFA gradient from 10:90:0.1 to 95:5:0.1 and acetonitrile/water/NH₄OH gradient from 10:90:0.1 to 95:5:0.1) to give the title compound (4 mg, 22%).

¹H-NMR (300 MHz, DMSO-d₆): δ 12.61 (s, 1H), 9.14 (s, 1H), 8.83 (s, 2H), 8.28 (d, J 2.2 Hz, 1H), 8.22 (d, J 2.4 Hz, 1H), 8.05 (d, J 2.2 Hz, 1H), 7.56 (dd, J 9.0, 2.6 Hz, 1H), 6.88 (d, J 9.2 Hz, 1H), 3.55 (m, 4H), 2.40 (m, 4H), 2.23 (s, 3H).

APCI-MS m/z=406.1 [MH⁻.

### EXAMPLE 69

### 5-Chloro-3-(4,5-dihydropyrimidin-5-yl)-2-(6-morpholin-4-ylpyridin-3-yl-1H-pyrrolo[2,3-b]pyridine

The title compound (4 mg, 17%) was synthesized from 5-chloro-2-(6-chloropyridin-3-yl)-3-pyrimidin-5-yl-1H-pyrrolo[2,3-b]pyridine (Example 59, 20 mg, 58 μmol) and morpholine (500 μl, 5.84 mmol) by the procedure of Example 68.

¹H-NMR (400 MHz, DMSO-d₆): δ 12.76 (d, J 115.8 Hz, 1H), 9.12 (s, 1H), 8.80 (d, J 5.4 Hz, 2H), 8.28 (d, J 2.1 Hz, 1H), 8.24 (d, J 2.4 Hz, 1H), 8.05 (d, J 2.2 Hz, 1H), 7.59 (dd, J 8.8, 2.6 Hz, 1H), 6.88 (d, J 8.8 Hz, 1H), 3.69 (t, J 4.8 Hz, 4H), 3.51 (t, J 4.9 Hz, 4H).

APCI-MS m/z=393.1 [MH⁺].

### EXAMPLE 70

### 1-[4-(5-Chloro-3-pyrimidin-5-yl-1H-pyrrolo[2,3-b]pyridin-2-yl)phenoxy]-3-(4-methylpiperazin-1-yl)propan-2-ol bis(trifluoroacetate)

5-Chloro-2-iodo-3-pyrimidin-5-yl-1H-pyrrolo[2,3-b]pyridine (prepared in analogy to Example 49a, 64 mg, 0.18 mmol), 1-(4-methylpiperazin-1-yl)-3-[4-(4,4,5,5-tetramethyl-1,3,2-dioxaborolan-2-yl)phenoxy]propan-2-ol (80 mg, 0.21 mmol),

1,1′-bis(diphenylphosphino)ferrocenedichloropalladium(II) (10 mg, 0.013 mmol) and potassium carbonate (87 mg, 0.63 mmol) were dissolved in dioxane (3 ml) and water (0.5 ml) and heated at 150° C. for 10 min in a microwave reactor (200 W) without cooling. The mixture was diluted with ethyl acetate and washed with water and brine. The organic layer was evaporated and the crude product was purified by preparative HPLC (RP-18, acetonitrile/water/TFA gradient from 10:90:0.1 to 95:5:0.1) to give the title compound (21 mg, 17%).

¹H-NMR (400 MHz, DMSO-d₆): δ 12.62 (s, 1H), 9.12 (s, 1H), 8.76 (s, 2H), 8.30 (d, J 2.2 Hz, 1H), 8.08 (d, J 2.2 Hz, 1H), 7.42 (d, J 8.7 Hz, 2H), 7.03 (d, J 8.8 Hz, 2H), 4.14 (s, 2H), 4.06-3.93 (m, 2H), 2.79 (s, 3H).

APCI-MS m/z: 479.1 [MH⁺].

### a) 1-(4-Methylpiperazin-1-yl)-3-[4-(4,4,5,5-tetramethyl-1,3,2-dioxaborolan-2-yl)phenoxy]propan-2-ol

4,4,5,5-Tetramethyl-2-(4-(oxiran-2-ylmethoxy)phenyl]-1,3,2-dioxaborolane (60 mg, 0.22 mmol) and N-methylpiperazine (34 ill, 0.31 mmol) were dissolved in isopropanol (2 ml) and heated to 100° C. for 10 min in a microwave reactor (100 W) without cooling. The reaction mixture was evaporated and the resulting oil was used directly in the next step.

APCI-MS m/z: 377.2 [MH⁺].

### b) 4,4,5,5-Tetramethyl-2-[4-(oxiran-2-ylmethoxy)phenyl]-1,3,2-dioxaborolane

2-[(4-Bromophenoxy)methyl]-oxirane (100 mg, 0.44 mmol), 4,4,4′,4′,5,5,5′,5′-octamethyl-2,2′-bi-1,3,2-dioxaborolane (153 mg, 0.66 mmol), potassium acetate (130 mg, 1.32 mmol) and 1,1′-bis(diphenylphosphino)ferrocenedichloropalladium(II) (11 mg, 0.013 mmol) were suspended in toluene (2 ml) and heated at 150° C. for 8 min in a microwave reactor without cooling. The mixture was diluted with ethyl acetate and washed with water and brine. The organic layer was purified by flash chromatography affording 62 mg (51%) of the subtitle compound as a colourless oil.

¹H-NMR (400 MHz, CDCl₃): δ 7.75 (d, J 8.6 Hz, 2H), 6.90 (d, J 8.6 Hz, 2H), 4.24 (dd, J 11.0, 3.2 Hz, 1H), 3.98 (dd, J 11.0, 5.6 Hz, 1H), 3.35 (dq, J 0.1, 3.0 Hz, 1H), 2.90 (t, J 4.5 Hz, 1H), 2.75 (dd, J 4.9, 2.6 Hz, 1H), 1.33 (s, 12H).

### c) 2-[(4-Bromophenoxy)methyl]-oxirane

p-Bromophenol (16.8 g, 97.0 mmol) was dissolved in THF (200 ml) and Cs₂CO₃ (37.8 g, 116 mmol) was added. Epibromohydrin (80 ml, 97 mmol) was added dropwise to the mixture. After heating to 50° C. for 25 h the mixture was poured into heptane/ethyl acetate (1:1), the salts were filtered off and the solvent was evaporated. This process was repeated once more to afford 21 g (95%) of the subtitle compound as a yellow oil.

¹H-NMR (400 MHz, CDCl₃): δ 7.34 (dd, J 6.7, 2.1 Hz, 2H), 6.74 (dd, J 6.7, 2.2 Hz, 2H), 3.49-3.35 (m, 2H), 3.35-3.29 (m, 1H), 2.99 (t, J 4.2 Hz, 1H), 2.72 (dd, J 4.7, 2.4 Hz, 1H).

### EXAMPLE 71

### 1-[4-(5-Chloro-3-pyrimidin-5-yl-1H-pyrrolo[2,3-b]pyridin-2-yl)phenoxy]-3-(dimethylamino)propan-2-ol bis(trifluoroacetate)

The title compound (16 mg, 18%) was prepared from 5-chloro-2-iodo-3-pyrimidin-5-yl-1H-pyrrolo[2,3-b]pyridine and dimethylamine (33% in ethanol) as described in Example 70.

¹H-NMR (400 MHz, DMSO-d₆): δ 12.59 (s, 1H), 9.11 (s, 1H), 8.76 (s, 2H), 8.29 (d, J 2.3 Hz, 1H), 8.07 (d, J 2.2 Hz, 1H), 7.39 (d, J 8.7 Hz, 2H), 7.00 (d, J 8.8 Hz, 2H), 4.01 (d, J 6.4 Hz, 1H), 3.96-3.86 (m, 2H), 2.34 (ddd, J 43.5, 12.4, 5.8 Hz, 2H), 2.20 (d, J 5.7 Hz, 6H).

APCI-MS m/z: 424.1 [MH⁺].

### EXAMPLE 72

### 1-[4-(5-Chloro-3-pyrimidin-5-yl-1H-pyrrolo[2,3-b]pyridin-2-yl)phenoxy]-3-morpholin-4-ylpropan-2-ol bis(trifluoroacetate)

The title compound (2 mg, 2%) was prepared from 5-chloro-2-iodo-3-pyrimidin-5-yl-1H-pyrrolo[2,3-b]pyridine and morpholine as described in Example 70.

¹H-NMR (400 MHz, DMSO-d₆): δ 12.60 (s, 1H), 9.12 (s, 1H), 8.76 (s, 2H), 8.29 (d, J 2.3 Hz, 1H), 8.07 (d, J 2.3 Hz, 1H), 7.40 (d, J 8.8 Hz, 2H), 7.01 (d, J 8.7 Hz, 2H), 4.90 (d, J 4.6 Hz, 1H), 4.17-3.87 (m, 4H), 3.56 (t, J 4.6 Hz, 4H), 2.47-2.27 (m, 4H). APCI-MS m/z: 466.1 [MH⁺].

### EXAMPLE 73

### 1-{3-[4-(5-Chloro-3-pyrimidin-5-yl-1H-pyrrolo[2,3-b]pyridin-2-yl)phenoxy]-2-hydroxypropyl}pyrrolidin-3-ol bis(trifluoroacetate)

The title compound (2 mg, 2%) was prepared from 5-chloro-2-iodo-3-pyrimidin-5-yl-1H-pyrrolo[2,3-b]pyridine and pyrrolidin-3-ol as described in Example 70.

¹H-NMR (400 MHz, DMSO-d₆): δ 12.59 (s, 1H), 9.11 (s, 1H), 8.76 (s, 2H), 8.29 (d, J 2.3 Hz, 1H), 8.07 (d, J 2.2 Hz, 1H), 7.39 (d, J 8.8 Hz, 2H), 7.01 (d, J 8.8 Hz, 2H), 4.87 (s, 1H), 4.64 (d, J 4.7 Hz, 1H), 4.23-4.09 (m, 2H), 4.10-3.97 (m, 2H), 3.89 (d, J 6.0 Hz, 2H), 2.05-1.90 (m, 2H), 1.60-1.42 (m, 2H).

APCI-MS m/z: 466.1 [MH⁺].

### EXAMPLE 74

### 1-(1,4′-Bipiperidin-1′-yl)-3-[4-(5-chloro-3-pyrimidin-5-yl-1H-pyrrolo[2,3-b]pyridin-2-yl)phenoxy]propan-2-ol bis(trifluoroacetate)

The title compound (5 mg, 5%) was prepared from 5-chloro-2-iodo-3-pyrimidin-5-yl-1H-pyrrolo[2,3-b]pyridine and 4-piperidinopiperidine as described in Example 70. APCI-MS m/z: 547.3 [MH⁺].

### EXAMPLE 75

### {3-[4-(5-Chloro-3-pyrimidin-5-yl-1H-pyrrolo[2,3-b]pyridin-2-yl)phenoxy]-2-methoxypropyl}dimethylamine

The title compound (16 mg, 26%) was synthesized from 5-chloro-2-iodo-3-pyrimidin-5-yl-1H-pyrrolo[2,3-b]pyridine and (2-methoxy-3-(4-(4,4,5,5-tetramethyl-1,3,2-dioxaborolan-2-yl)phenoxy]propyl)dimethylamine as described in Example 70. Purification was performed by preparative HPLC (acetonitrile/water/NH₄OH gradient from 10:90:0.2 to 95:5:0.2).

¹H-NMR (400 MHz, DMSO-d₆): δ 12.58 (s, 1H), 9.10 (s, 1H), 8.76 (s, 2H), 8.28 (d, J 2.3 Hz, 1H), 8.05 (d, J 2.3 Hz, 1H), 7.40 (d, J 8.8 Hz, 2H), 7.01 (d, J 8.8 Hz, 2H), 4.13 (dd, J 10.4, 3.4 Hz, 1H), 4.00 (dd, J 10.4, 5.6 Hz, 1H), 3.61 (dt, J 9.2, 5.8 Hz, 1H), 3.36 (s, 3H), 2.47-2.30 (m, 2H), 2.18 (s, 6H).

APCI-MS m/z: 438.1 [MH⁺].

### a) {2-Methoxy-3-[4-(4,4,5,5-tetramethyl-1,3,2-dioxaborolan-2-yl)phenoxy]propyl}dimethylamine

[3-(4-Bromophenoxy)-2-methoxypropyl]dimethyl amine (530 mg, 1.84 mmol), 4,4,4′,4′,5,5,5′,5′-octamethyl-2,2′,bi-1,3,2-dioxaborolane (701 mg, 2.76 mmol), potassium acetate (542 mg, 5.52 mmol) and 1,1′-bis(diphenylphosphino)ferrocenedichloro-palladium(II) (45 mg, 0.055 mmol) were suspended in toluene (12 ml) and divided into 3 microwave vials. Each vial was heated at 150° C. for 13 min in a microwave reactor (250 W) without cooling. The combined mixtures were diluted with ethyl acetate and washed with water and brine. The organic layer was evaporated and crude product purified by preparative HPLC (RP-18, CH₃CN/water+0.1% TFA). The compound was dissolved in saturated sodium carbonate solution and extracted with ethyl acetate to afford the subtitle compound as a slightly pink oil (47 mg, 8%).

¹H-NMR (400 MHz, CDCl₃): δ 7.75 (d, J 8.7 Hz, 2H), 6.92 (d, J 28.9 Hz, 2H), 4.13 (dt, J 14.0, 3.7 Hz, 1H), 4.03 (dd, J 10.1, 5.1 Hz, 1H), 3.76 (quintet, J 5.2 Hz, 1H), 3.52 (s, 3H), 2.66-2.61 (m, 2H), 2.39 (s, 6H), 1.36 (s, 12H)

APCI-MS m/z: 336.2 [MH⁺].

### b) [3-(4-Bromophenoxy)-2-methoxypropyl]dimethylamine

1-(4-Bromophenoxy)-3-(dimethylamino)propan-2-ol (580 mg, 2.11 mmol) was dissolved in THF (10 ml) and sodium hydride was added (253 mg, 10.55 mmol, 60% in oil). After 10 min. iodomethane (105 μl, 2.53 mmol) was added and the mixture was stirred at room temperature for 4 h. The reaction was quenched with water and the volume reduced by rotary evaporation. The residue was partitioned between water and ethyl acetate. The organic layer was washed with brine, dried over sodium sulfate and the solvent was removed to afford the crude subtitle compound as a yellow oil (533 mg, 88%)

¹H-NMR (400 MHz, CDCl₃): δ 7.38 (d, J 40.2 Hz, 2H), 6.81 (dd, J 6.9, 2.1 Hz, 2H), 4.08 (dd, J 0.0, 3.9 Hz, 1H), 3.98 (dd, J 10.1, 5.1 Hz, 1H), 3.76 (s, 1H), 3.50 (d, J 2.2 Hz, 3H), 2.61 (d, J 6.3 Hz, 2H), 2.38 (s, 6H)

APCI-MS m/z: 288.1, 290.2 [MH⁺]).

### c) 1-(4-bromophenoxy)-3-(dimethylamino)propan-2-ol

2-[(4-Bromophenoxy)methyl]-oxirane (500 mg, 2.18 mmol) and dimethylamine (33% in ethanol, 4 ml) were dissolved in isopropanol (5 ml) and poured into two microwave vials. Each vial was heated at 100 CC for 10 min in a microwave reactor (100 W, 100 psi) without cooling. The reaction mixtures were combined and evaporated and the resulting oil (588 mg, 98%) was used directly in the next step.

APCI-MS m/z: 274.2, 276.1 [MH⁺].

### EXAMPLE 76

### [4-(5-Chloro-3-pyrimidin-5-yl-1H-pyrrolo[2,3-b]pyridin-2-yl)phenyl][2-(4-methylpiperazin-1-yl)ethyl]amine

A solution of (2-chloroethyl)[4-(5-chloro-3-pyrimidin-5-yl-1H-pyrrolo[2,3-b]pyridin-2-yl)phenyl]amine in NMP (3 ml) and N-methylpiperazine (1 ml) was heated to 140° C. for 15 min in a microwave reactor (250 W) without cooling. The mixture was partitioned between water and ethyl acetate, the organic layer washed with brine, evaporated and purified by preparative HPLC [acetonitrile/water (0.1% TFA)] to yield the title compound (80 mg, 64%).

¹H-NMR (400 MHz, DMSO-d₆): δ 12.42 (s, 1H), 9.10 (s, 1H), 8.77 (s, 2H), 8.24 (d, J 2.2 Hz, 1H), 8.00 (d, J 2.1 Hz, 1M), 7.23 (d, J 8.6 Hz, 2H), 6.64 (d, J 8.6 Hz, 2H), 3.60-2.84 (m, 12H), 2.79 (s, 3H).

APCI-MS m/z: 448.2 [MH⁺].

### a) (2-Chloroethyl)[4-(5-chloro-3-pyrimidin-5-yl-1H-pyrrolo[2,3-b]pyridin-2-yl)phenyl]amine

[4-(5-Chloro-3-pyrimidin-5-yl-1H-pyrrolo[2,3-b]pyridin-2-yl)phenyl]amine (90 mg, 0.28 mmol) and sodium cyanoborohydride were dissolved in NMP (3.5 ml) and acetonitrile (4.5 ml). Chloroacetaldehyde (45% in water, 50 μl, 0.76 mmol) and some MgSO₄ were added and the pH adjusted to approx. 4 with conc. H₂SO₄. The suspension was stirred at room temperature overnight after which it was filtered through a plug of celite. The volatiles were removed by rotary evaporation and the resulting solution was used directly.

APCI-MS m/z: 384.1, 386.2 [MH⁺].

### b) [4-(5-Chloro-3-pyrimidin-5-yl-1H-pyrrolo[2,3-b]pyridin-2-yl)phenyl]amine

tert-Butyl[4-(5-chloro-3-pyrimidin-5-yl-1H-pyrrolo[2,3-b]pyridin-2-yl)phenyl]carbamate (180 mg, 0.43 mmol) was dissolved in dichloromethane (10 ml) and trifluoroacetic acid (10 ml). The solution was stirred at room temperature for 1 h after which the solvent was removed by rotary evaporation. The residue was suspended in water and basified with saturated sodium carbonate. The solution was extracted several times with dichloromethane and the combined organic phases were evaporated affording the title compound as a yellow powder (55 mg, 40%).

APCI-MS m/z: 322.2 [MH⁺].

### c) tert-Butyl[4-(5-chloro-3-pyrimidin-5-yl-1H-pyrrolo[2,3-b]pyridin-2-yl)phenyl]carbamate

5-Chloro-2-iodo-3-pyrimidin-5-yl-1H-pyrrolo[2,3-b]pyridine (prepared by analogy to Example 49a, 200 mg, 0.56 mmol), tert-butyl[4(4,4,5,5-tetramethyl-1,3,2-dioxaborolan-2-yl)phenyl]carbamate (268 mg, 0.84 mmol), 1,1′-bis(diphenylphosphino)ferrocene-dichloropalladium(II) (28 mg, 0.034 mmol) and potassium carbonate (232 mg, 1.68 mmol) were dissolved in dioxane (10 ml) and water (1 ml) and heated to 90° C. for 48 h. The mixture was poured onto an acidic ion exchange resin (Dowex 50WX2) and stirred for 10 min. The resin was filtered off and washed successively with methanol and 5% ammonia in methanol. The filtrate was evaporated affording the title compound as a greenish powder (190 mg, 81%).

¹H-NMR (400 MHz, DMSO-d₆): δ 12.59 (s, 1H), 9.56 (s, 1H), 9.11 (s, 1H), 8.75 (s, 2H), 8.29 (d, J 2.2 Hz, 1H), 8.07 (d, J 2.3 Hz, 1H), 7.50 (d, J 8.7 Hz, 2H), 7.36 (d, J 8.7 Hz, 2H), 1.48 (s, 9H).

APCI-MS m/z: 422.1 [MH⁺].

### EXAMPLE 77

### 5-Chloro-2-(1H-pyrazol-4-yl)-3-pyridin-3-yl-1H-pyrrolo[2,3-b]pyridine

The title compound (87 mg, 33%) was prepared from 5-chloro-2-iodo-3-pyridin-3-yl-1H-pyrrolo[2,3-b]pyridine (150 mg, 0.42 mmol) and 4-(4,4,5,5-tetramethyl-1,3,2-dioxaborolan-2-yl)-1H-pyrazole by a procedure similar to Example 49.

¹H-NMR (300 MHz, DMSO-d₆): δ 12.50 (s, 1H), 8.86 (d, J 1.6 Hz, 1H), 8.75 (dd, J 5.3, 1.3 Hz, 1H), 8.30 (dt, J 8.0, 1.8 Hz, 2H), 8.25 (d, J 2.4 Hz, 1H), 7.95 (d, J 2.2 Hz, 1H), 7.84 (dd, J 8.1, 5.2 Hz, 3H).

APCI-MS m/z: 296.1 [MH⁺].

### EXAMPLE 78

### 5-Chloro-2-{4-[3-(dimethylamino)propoxy]phenyl}-N-methyl-3-pyrimidin-5-yl-1H-pyrrolo[2,3-b]pyridin-4-amine,

The title compound (5 mg, 10%) was synthesized from 5-chloro-3-iodo-N-4-methylpyridine-2,4-diamine (42 mg, 0.11 mmol) by a procedure similar to Example 49.

¹H-NMR (300 MHz, DMSO-d₆): δ 12.26 (s, 1H), 9.11 (s, 1H), 8.67 (s, 2H), 8.04 (s, 1H), 7.20 (d, J 8.8 Hz, 2H), 6.92 (d, J 8.7 Hz, 2H), 5.23 (d, J 5.6 Hz, 1H), 4.00 (t, J 6.4 Hz, 2H), 2.34 (t, J 7.1 Hz, 2H), 2.23 (d, J 5.5 Hz, 3H), 2.13 (s, 6H), 1.83 (quintet, J 6.7 Hz, 2H).

APCI-MS m/z: 437.1 [MH⁺].

### Screen

Itk LANCE TRF Assay

The Itk kinase assay utilized recombinant human Itk kinase domain fused with GST (Glutathione S-Transferase). The protein was expressed in High five insect cells, purified in one step on an affinity chromatography glutathione column and stored in 50 mM Tris/HCl (pH 7.6), 150 mM NaCl, 5% (w/v) mannitol, 1 mM DTT, 30% glycerol at −70° C. The kinase substrate used in the assay was a biotinylated peptide derived from the Src-optimal substrate (Nair et al, J. Med. Chem., 38: 4276, 1995; biotin-AEEEIYGEFEAKKKK).

The assay additions were as follows: Test compounds (or controls; 1 μL in 100% DMSO) were added to black 96-well flat-bottomed plates (Greiner 655076) followed by 20 μL Itk in assay buffer and the reaction was started by adding 20 μL ATP and peptide substrate in assay buffer. The assay buffer constitution during phosphorylation was: 50 mM HEPES (pH 6.8), 10 mM MgCl₂, 0.015% Brij 35, 1 mM DTT, 10% glycerol, 160 ng/well Itk, 2 μM peptide substrate and 50 μM ATP. The assay was stopped after 50 minutes (RT) by adding 150 μL ice-cold Stop solution (50 mM Tris/HCl, pH 7.5, 10 mM EDTA, 0.9% NaCl and 0.1% BSA) together with LANCE reagents (2 nM PT66-Eu³⁺, Wallac AD0069 and 5 μg/ml Streptavidin-APC, Wallac AD0059. Both concentrations were final in stopped assay solution). The plates were measured on a Wallac 1420 Victor 2 instrument with TRF settings after 1 h incubation, and the ratio (665 signal/615 signal)*10000 was used to calculate the inhibition values. IC₅₀ values were determined using XLfit.

When tested in the above screens, the compounds of Examples 1 to 78 gave IC₅₀ values for inhibition of Itk activity of less than 25 μM, indicating that the compounds of the invention are expected to possess useful therapeutic properties.

Representative results are shown in the following Table:

| | Inhibition of |
|---|---|
| Compound | Kinase Itk (IC₅₀ μM) |
| Example 5 | 0.01 |
| Example 13 | 0.03 |
| Example 44 | 0.44 |
| Example 55 | 0.01 |
| Example 61 | 0.04 |

1. A compound of formula (I): 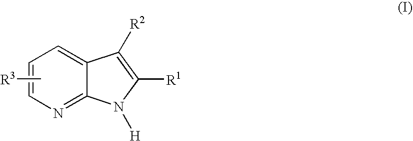
wherein:
R¹ represents phenyl or a five or six membered aromatic heterocyclic ring containing 1 to 3 heteroatoms selected independently from O, S and N; said phenyl or aromatic heterocyclic ring being optionally substituted by one or more substituents selected independently from halogen, C1 to 4 alkyl, C1 to 4 alkoxy, CO₂R⁴ or a group—Q-L-M;
Q represents CO, O, NR¹² or a bond;
L represents C1 to 4 alkyl optionally further substituted by OH or OMe; or L represents a bond;
M represents NR¹³R¹⁴ or OR¹⁵;
R¹³ and R¹⁴ independently represent H, C1 to 4 alkyl or CONH₂; or the group —NR¹³R¹⁴ together represents a saturated 5 to 7 membered azacyclic ring optionally incorporating one further heteroatom selected from O, S and NR¹⁶ and optionally further substituted by OH or 1-piperidinyl;
R¹⁶ represents H, C1 to 4 alkyl, CHO or C2 to 4 alkanoyl;
R² represents phenyl or a five or six membered aromatic heterocyclic ring containing 1 to 3 heteroatoms independently selected from O, S and N; said phenyl or aromatic heterocyclic ring being optionally substituted by one or more substituents selected independently from halogen, C1 to 4 alkyl, C1 to 4 alkoxy, OH, CN, CO₂R⁶ and a group —W—X—Y;
W represents O or a bond;
X represents C1 to 4 alkyl, —CO—, —CH₂CHOHCH₂— or a bond;
Y represents NR⁷R⁸;
or Y represents a saturated or partially unsaturated 4 to 7 membered ring, optionally including 1 or 2 heteroatoms independently selected from O, N and S(O)ₙ and optionally incorporating 1 or 2 carbonyl groups; and optionally substituted by one or more substituents selected independently from OH, C1 to 4 alkyl, C1 to 4 alkoxy, CHO, C2 to 4 alkanoyl, C1 to 4 alkylsulphonyl or CO₂R⁵;
or Y represents C1 to 4 alkoxy optionally further substituted by OH or C1 to 4 alkoxy;
R³ represents H or one or two substituents selected independently from halogen, C1 to 4 alkyl, C1 to 4 alkoxy or cyano;
R⁴, R⁵ and R⁶ independently represent H or C1 to 4 alkyl;
R⁷ and R⁸ independently represent H, C1 to 4 alkyl, —CH₂CHOHCH₂OH, CHO, C2 to 4 alkanoyl or a group —G-J-K wherein G represents —CO— or a bond; J represents C1 to 4 alkyl;
and K represents —NR⁹R¹⁰ or —CH(NH₂)CO₂R¹¹;
R⁹ and R¹⁰ independently represent H or C1 to 4 alkyl; or the group —NR⁹R¹⁰ together represents a saturated 5 or 6 membered azacyclic ring;
R¹¹, R¹² and R¹⁵ independently represent H or C1 to 4 alkyl;
n represents an integer 0, 1 or 2;
and pharmaceutically acceptable salts thereof;
provided that:
(i) when R³ is at the 6-position and represents C1 to 4 alkoxy and at the same time R¹ represents optionally substituted phenyl, then R² does not represent unsubstituted 4-pyridyl or unsubstituted 4-pyrimidyl; and
(ii) when R² represents 4-hydroxyphenyl or 4-hydroxy-3-pyridyl either optionally further substituted by halogen, C1 to 4 alkyl or C1 to 4 alkoxy, then R³ represents cyano; and
(iii) the following three compounds are disclaimed -2-(4-fluorophenyl)-3-(4-pyridinyl)-1H-pyrrolo[2,3-b]pyridine; 2,3-diphenyl-1H-pyrrolo[2,3-b]pyridine; and 4-methyl-2,3-diphenyl-1H-pyrrolo[2,3-b]pyridine.

2. A compound according to claim 1 wherein R³ represents halogen, methyl, methoxy or cyano.

3. A compound according to claim 1 wherein R² represents phenyl substituted by a group —W—X—Y and W represents O.

4. A compound of formula (I), according to claim 1, which is:
5-bromo-3-(4-methoxyphenyl)-2-phenyl-1H-pyrrolo[2,3-b]pyridine;
-bromo-3-(3-methoxyphenyl)-2-phenyl-1H-pyrrolo[2,3-b]pyridine;
4-(5-bromo-2-phenyl-1H-pyrrolo[2,3-b]pyridin-3-yl)benzonitrile;
5-bromo-2-(2-furyl)-3-phenyl-1H-pyrrolo[2,3-b]pyridine;
3-{4-[5-bromo-2-(2-furyl)-1H-pyrrolo[2,3-b]pyridin-3-yl]phenoxy}-N,N-dimethylpropan-1-amine;
5-bromo-3-(4-morpholin-4-ylphenyl)-2-phenyl-1H-pyrrolo[2,3-b]pyridine;
5-bromo-2,3-diphenyl-1H-pyrrolo[2,3-b]pyridine;
5-bromo-2-(4-bromophenyl)-3-phenyl-1H-pyrrolo[2,3-b]pyridine;
5-bromo-2,3-bis(4-methoxyphenyl)-1H-pyrrolo[2,3-b]pyridine;
N-(3-{4-[5-bromo-2-(2-furyl)-1H-pyrrolo[2,3-b]pyridin-3-yl]phenoxy}propyl)-N,N-dimethylamine;
5-bromo-3-phenyl-2-(1,3-thiazol-2-yl)-1H-pyrrolo[2,3-b]pyridine;
5-bromo-3-furan-2-yl-1-H-pyrrolo[2,3-b]pyridine;
N-[5-(5-bromo-2-phenyl-1-H-pyrrolo[2,3-b]pyridin-3-yl)-fuan-2-ylmethyl]-acetamide;
5-bromo-3-(5-aminomethylfuran-2-yl)-2-phenyl-1H-pyrrolo[2,3-b]pyridine;
5-bromo-2,3-difuran-2-yl-1H-pyrrolo[2,3-b]pyridine;
methyl 5-(5-bromo-3-phenyl-1H-pyrrolo[2,3-b]pyridin-2-yl)-1H-pyrrole-2-carboxylate;
5-bromo-3-phenyl-2-(1H-pyrrol-3-yl)-1H-pyrrolo[2,3-b]pyridine;
5-bromo-3-phenyl-2-(1,3-oxazol-2-yl)-1H-pyrrolo[2,3-b]pyridine;
3-(5-bromo-2-phenyl-1H-pyrrolo[2,3-b]pyridin-3-yl)phenol;
1-[4-(5-bromo-2-phenyl-1H-pyrrolo[2,3-b]pyridin-3-yl)phenoxy]-3-[(2-pyrrolidin-1-ylethyl)amino]propan-2-ol;
1-[4-(5-bromo-2-phenyl-1H-pyrrolo[2,3-b]pyridin-3-yl)phenoxy]-3-pyrrolidin-1-ylpropan-2-ol;
5-bromo-3-{4-[2-(1-methylpyrrolidin-2-yl)ethoxy]phenyl}-2-phenyl-1H-pyrrolo[2,3-b]pyridine;
5-bromo-2-phenyl-3-[4-(2-pyrrolidin-1-ylethoxy)phenyl]-1H-pyrrolo[2,3-b]pyridine;
5-bromo-3-[4-(2-morpholin-4-ylethoxy)phenyl]-2-phenyl-1H-pyrrolo[2,3-b]pyridine;
-bromo-3-[3-(2-morpholin-4-ylethoxy)phenyl]-2-phenyl-1H-pyrrolo[2,3-b]pyridine;
3-[4-(5-bromo-2-phenyl-1H-pyrrolo[2,3-b]pyridin-3-yl)phenoxy]-N,N-dimethylpropan-1-amine;
5-bromo-3-{4-[2-(2-methoxyethoxy)ethoxy]phenyl}-2-phenyl-1H-pyrrolo[2,3-b]pyridine;
5-bromo-3-{3-[2-(1-methylpyrrolidin-2-yl)ethoxy]phenyl}-2-phenyl-1H-pyrrolo[2,3-b]pyridine;
3-{4-[3-(dimethylamino)propoxy]phenyl}-2-phenyl-1H-pyrrolo[2,3-b]pyridine-5-carbonitrile;
5-{[4-(5-bromo-2-phenyl-1H-pyrrolo[2,3-b]pyridin-3-yl)phenoxy]methyl}-1,3-oxazolidin-2-one;
3-{4-[3-(dimethylamino)propoxy]phenyl}-2-(4-methoxyphenyl)-1H-pyrrolo[2,3-b]pyridine-5-carbonitrile;
(3-{4-[5-bromo-2-(4-methoxy-phenyl)-1H-pyrrolo[1,3-b]pyridin-3-yl]-phenoxy}-propyl)-dimethylamine;
3-[4-(5-bromo-2-phenyl-1H-pyrrolo[2,3-b]pyridin-3-yl)phenoxy]propan-1-amine;
5-bromo-3-(4-aminomethylphenyl)-2-phenyl-1H-pyrrolo[2,3-b]pyridine;
5-bromo-3-[4-(4,5-dihydro-1H-imidazol-2-yl)phenyl]-2-phenyl-1H-pyrrolo[2,3-b]pyridine;
5-bromo-3-[4-(4,4-dimethyl-4,5-dihydro-1H-imidazol-2-yl)phenyl]-2-phenyl-1H-pyrrolo[2,3-b]pyridine;
N-(2-aminoethyl)-4-(5-bromo-2-phenyl-1H-pyrrolo[2,3-b]pyridin-3-yl)benzamide;
3-[[4-(5-bromo-2-phenyl-1H-pyrrolo[2,3-b]pyridin-3-yl)benzyl](1,2-dihydroxypropyl)amino]propane-1,2-diol;
4-(5-bromo-2-phenyl-1H-pyrrolo[2,3-b]pyridin-3-yl)benzoic acid;
N⁵-[4-(5-bromo-2-phenyl-1H-pyrrolo[2,3-b]pyridin-3-yl)benzyl]glutamine;
3-(4-hydroxyphenyl)-2-phenyl-1H-pyrrolo[2,3-b]pyridine-5-carbonitrile;
3-[4-(aminomethyl)phenyl]-2-phenyl-1H-pyrrolo[2,3-b]pyridine-5-carbonitrile;
3-(4-morpholin-4-ylphenyl)-2-phenyl-1H-pyrrolo[2,3-b]pyridine-5-carbonitrile;
3-(4-hydroxyphenyl)-2-(4-methoxyphenyl)-1H-pyrrolo[2,3-b]pyridine-5-carbonitrile;
5-bromo-2-phenyl-3-pyrrol-1-yl-1H-pyrrolo[2,3-b]pyridine;
5-cyano-2-(4-methoxy-phenyl)-3-pyrrol-1-yl-1H-pyrrolo[2,3-b]pyridine;
5-bromo-3-(2,5-dimethyl-pyrrol-1-yl)-2-phenyl-1H-pyrrolo[2,3-b]pyridine;
3-(4-methoxyphenyl)-2-phenyl-1H-pyrrolo[2,3-b]pyridine-5-carbonitrile;
{3-[4-(5-methyl-3-pyrimidin-5-yl-1H-pyrrolo[2,3-b]pyridin-2-yl)phenoxy]propyl}dimethylamine;
{3-[4-(5-fluoro-3-pyrimidin-5-yl-1H-pyrrolo[2,3-b]pyridin-2-yl)phenoxy]propyl}dimethylamine;
2-{4-[3-(dimethylamino)propoxy]phenyl}-4-methyl-3-pyridin-3-yl-1H-pyrrolo[2,3-b]pyridine-5-carbonitrile;
5-chloro-2-[5-(piperazin-1-ylcarbonyl)-1H-pyrrol-3-yl]-3-pyridin-3-yl-1H-pyrrolo[2,3-b]pyridine;
5-chloro-2-[5-(piperazin-1-ylcarbonyl)-1H-pyrrol-3-yl]-3-pyrimidin-5-yl-1H-pyrrolo[2,3-b]pyridine;
{3-[4-(4,5-dichloro-3-pyrimidin-5-yl-1H-pyrrolo[2,3-b]pyridin-2-yl)phenoxy]propyl}dimethylamine;
{3-[4-(5-bromo-4-methyl-3-pyridin-3-yl-1H-pyrrolo[2,3-b]pyridin-2-yl)phenoxy]propyl}dimethylamine;
5-chloro-3-pyridin-3-yl-2-(1H-pyrrol-2-yl)-1H-pyrrolo[2,3-b]pyridine;
5-chloro-3-pyridin-3-yl-2-(1H-pyrrol-3-yl)-1H-pyrrolo[2,3-b]pyridine;
5-chloro-4-methoxy-3-pyridin-3-yl-2-(1H-pyrrol-3-yl)-1H-pyrrolo[2,3-b]pyridine;
5-chloro-2-(6-chloropyridin-3-yl)-3-pyrimidin-5-yl-1H-pyrrolo[2,3-b]pyridine;
(2-{[5-(5-chloro-3-pyrimidin-5-yl-1H-pyrrolo[2,3-b]pyridin-2-yl)pyridin-2-yl]oxy}ethyl)methylamine;
N-[5-(5-chloro-3-pyrimidin-5-yl-1H-pyrrolo[2,3-b]pyridin-2-yl)pyridin-2-yl]-N,N′,N′-trimethylpropane-1,3-diamine;
N′-[5-(5-chloro-3-pyrimidin-5-yl-1H-pyrrolo[2,3-b]pyridin-2-yl)pyridin-2-yl]-N,N-dimethylpropane-1,3-diamine;
N-{3-[4-(5-chloro-3-pyrimidin-5-yl-1H-pyrrolo[2,3-b]pyridin-2-yl)phenoxy]propyl}-N,N-dimethylamine;
{3-[4-(5-chloro-4-methoxy-3-pyridin-3-yl-1H-pyrrolo[2,3-b]pyridin-2-yl)phenoxy]propyl}dimethylamine;
N-(2-{[5-(5-chloro-3-pyrimidin-5-yl-1H-pyrrolo[2,3-b]pyridin-2-yl)pyridin-2-yl]oxy}ethyl)urea;
2-{[5-(5-chloro-3-pyrimidin-5-yl-1H-pyrrolo[2,3-b]pyridin-2-yl)pyridin-2-yl]oxy}ethanol;
2-[6-(4-acetylpiperazin-1-yl)pyridin-3-yl]-5-chloro-3-pyrimidin-5-yl-1H-pyrrolo[2,3-b]pyridine;
5-chloro-3-(4,5-dihydropyrimidin-5-yl)-2-[6-(4-methylpiperazin-1-yl)pyridin-3-yl]-1H-pyrrolo[2,3-b]pyridine;
5-chloro-3-(4,5-dihydropyrimidin-5-yl)-2-(6-morpholin-4-ylpyridin-3-yl)-1H-pyrrolo[2,3-b]pyridine;
1-[4-(5-chloro-3-pyrimidin-5-yl-1H-pyrrolo[2,3-b]pyridin-2-yl)phenoxy]-3-(4-methylpiperazin-1-yl)propan-2-ol;
1-[4-(5-chloro-3-pyrimidin-5-yl-1H-pyrrolo[2,3-b]pyridin-2-yl)phenoxy]-3-(dimethylamino)propan-2-ol;
1-[4-(5-chloro-3-pyrimidin-5-yl-1H-pyrrolo[2,3-b]pyridin-2-yl)phenoxy]-3-morpholin-4-ylpropan-2-ol;
1-{3-[4-(5-chloro-3-pyrimidin-5-yl-1H-pyrrolo[2,3-b]pyridin-2-yl)phenoxy]-2-hydroxypropyl}pyrrolidin-3-ol;
1-(1,4′-bipiperidin-1′-yl)-3-[4-(5-chloro-3-pyrimidin-5-yl-1H-pyrrolo[2,3-b]pyridin-2-yl)phenoxy]propan-2-ol;
{3-[4-(5-chloro-3-pyrimidin-5-yl-1H-pyrrolo[2,3-b]pyridin-2-yl)phenoxy]-2-methoxypropyl}dimethylamine;
[4-(5-chloro-3-pyrimidin-5-yl-1H-pyrrolo[2,3-b]pyridin-2-yl)phenyl][2-(4-methylpiperazin-1-yl)ethyl]amine;
5-chloro-2-(1H-pyrazol-4-yl)-3-pyridin-3-yl-1H-pyrrolo[2,3-b]pyridine;
5-chloro-2-{4-[3-(dimethylamino)propoxy]phenyl}-N-methyl-3-pyrimidin-5-yl-1H-pyrrolo[2,3-b]pyridin-4-amine;
or a pharmaceutically acceptable salt of any one thereof.

5. (canceled)

6. A pharmaceutical formulation comprising a compound of formula (I), as defined in claim 1, or a pharmaceutically acceptable salt thereof, optionally in admixture with a pharmaceutically acceptable diluent or carrier.

7. A method of treating, or reducing the risk of, a human disease or condition in which inhibition of Itk kinase activity is beneficial which comprises administering to a person suffering from or susceptible to such a disease or condition, a therapeutically effective amount of a compound of formula (I), as defined in claim 1, or a pharmaceutically acceptable salt thereof.

8. (canceled)

9. The method according to claim 7 wherein the disease is asthma.

10. The method according to claim 7 wherein the disease is allergic rhinitis.

11. A process for the preparation of a compound of formula (I), as defined in claim 1, and optical isomers and racemates thereof and pharmaceutically acceptable salts thereof, which comprises:
a) reaction of a compound of formula (II): 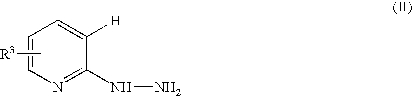
in which R³ is as defined in claim 1, with a compound of formula (III): 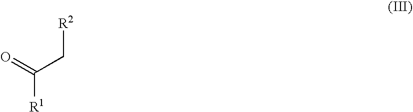
in which R¹ and R² are as defined in claim 1; or
b) arylation of a compound of formula (IV) 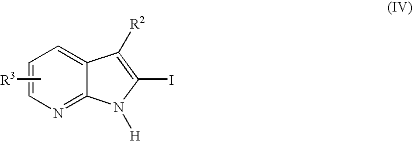
wherein R² and R³ are as defined in claim 1, with a boronic acid of formula R¹—B(OH)₂ wherein R¹ is as defined in claim 1;
and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.
